(19) United States
(12) Patent Application Publication
(10) Pub. No.: US 2002/0137750 A1
(43) Pub. Date: Sep. 26, 2002

(54) Amidine derivatives which are inhibitors of nitric oxide synthase

(76) Inventors: D.W. Chen Deborah, Wilmington, DE (US); James R. Empfield, Wilmington, DE (US); James E. Macdonald, San Diego, CA (US); Kenneth C. Mattes, Marlboro, MA (US); Robert Murray, Waltham, MA (US); Eifion Phillips, Greenville, DE (US); Hans Schmitthenner, Rush, NY (US)

Correspondence Address: Nixon & Vanderhye, 1100 North Glebe Road 8th Floor, Arlington, VA, 22201-4714, US

(21) Appl. No.: 09/763,838

(22) PCT Filed: Dec. 14, 2000
(86) PCT No.: PCT/SE99/02540

(30) Foreign Application Priority Data
Dec. 20, 1999 (SE) 9904677-3

(51) Int. Cl. C 07D 4 9/02; A61K 31/496; A61K 31/4523; A61K 31/4025; A61K 31/381

(52) U.S. Cl. 514/252.13; 514/422; 514/448; 514/326; 544/374; 546/212; 548/527; 549/27

(57) ABSTRACT

There are provided novel compounds of formula (I)

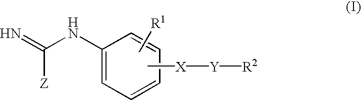

wherein R¹, R², X, Y and Z are as defined in the Specification and optical isomers, racemates and tautomers thereof and pharmaceutically acceptable salts thereof; tocether with processes for their preparation, compositions containing them and their use in therapy. The compounds are inhibitors of the enzyme nitric oxide synthase.

### FIELD OF THE INVENTION

This invention relates to new amidine derivatives, processes for their preparation, compositions containing them and their use in therapy.

### BACKGROUND OF THE INVENTION

Nitric oxide is produced in mammalian cells from L-arginine by the action of specific nitric oxide synthases (NOSs). These enzymes fall into two distinct classes—constitutive NOS (cNOS) and inducible NOS (iNOS). At the present time, two constitutive NOSs and one inducible NOS have been identified. Of the constitutive NOSs, an endothelial enzyme (ecNOS) is involved with smooth muscle relaxation and the regulation of blood pressure and blood flow, whereas the neuronal enzyme (ncNOS) serves as a neurotransmitter and appears to be involved in the regulation of various biological functions such as cerebral ischaemia. Inducible NOS has been implicated in the pathogenesis of inflammatory diseases. Specific regulation of these enzymes should therefore offer considerable potential in the treatment of a wide variety of disease states.

Considerable effort has been expended in efforts to identify compounds that act as specific inhibitors of one or more isoforms of the enzyme nitric oxide synthase. The use of such compounds in therapy has also been widely claimed.

WO 95/05363discloses compounds of generic structure

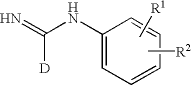

wherein D represents an aromatic ring, R¹ represents hydrogen, alkyl C1 to 6 or halogen; and R² represents a variety of nitrogen containing side-chains. The compounds have nitric oxide synthase inhibitory activity.

It has now surprisingly been found that a group of compounds with similar structures but which are not within the generic scope of WO 95/05363 possess unexpectedly advantageous properties. Such compounds are the subject of the present application.

### DISCLOSURE OF THE INVENTION

According to the invention we provide a compound of formula (I)

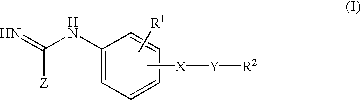

wherein

Z represents a furan or thiophene ring, optionally substituted by one or more substituents selected from halogen, trifluoromethyl, C1 to 6 alkyl, C1 to 6 alkoxy, hydroxy, amino, S(O)_{q}R⁴, CO₂R⁵ and CONR⁶R⁷;

X represents C1 to 6 alkyl;

Y represents O, S(O)ₙ or NR³;

n and q independently represent an integer 0, 1 or 2;

R¹ represents hydrogen, halogen, C1 to 6 alkyl, hydroxy, C1 to 6 alkoxy,

C1 to 6 alkoxy-O—R⁸, C1 to 6 alkoxy-NR⁹R¹⁰ or —O-phenyl; said phenyl being optionally substituted by one or more substituents selected from halogen, trifluoromethyl, C1 to 6 alkyl, C1 to 6 alkoxy, hydroxy and amino;

R² represents C1 to 6 alkyl-O—R¹¹ or C1 to 6 alkyl-NR¹²R¹³;

R³ represents hydrogaen, C1 to 6 alkyl, C2 to 7 alkanoyl, C1 to 6 alkyl-O—R¹⁴,

C1 to 6 alkyl-NR¹⁵R¹⁶ or —CH₂-phenyl; said phenyl being optionally substituted by one or more substituents selected Mom halogen, trifluoromethyl, C1 to 6 alkyl, C1 to 6 alkoxy, hydroxy and amino;

or the group NR²R³ represents azetidinyl, pyrrolidinyl, piperidinyl, morpholinyl; or

piperazinyl optionally 4-substituted by C1 to 6 alkyl; each of said azacyclic rings being substituted by OR—R¹⁷, NR¹⁸R¹⁹, C1 to 6 alkyl-O—R¹⁷ or C1 to 6 alkyl-NR¹⁸R¹⁹;

or, when Y represents NR³, the groups X and R³ are joined together such that the group X—N—R³ represents a saturated 4 to 7 membered azacyclic ring;

R⁴, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ independently represent hydrogen or C1 to 6 alkyl;

or the groups NR⁹R¹⁰, NR¹²R¹³, NR¹⁵R¹⁶ and NR¹⁸R¹⁹ independently represent azetidinyl, pyrrolidinyl, piperidinyl, morpholinyl; or piperazinyl optionally 4-substituted by C1 to 6 alkyl;

and optical isomers, racemates and tautomers thereof and pharmaceutically acceptable salts thereof.

In one preferred embodiment, Z represents unsubstituted 2-thienyl or 3-thienyl. More preferably 2-thienyl.

Preferably the substituent R¹ in formula (I) is in the ortho or para position relative to the amidine group. More preferably the substituent R¹ in formula (I) is in the para position relative to the amidine group, as shown in formula (IA).

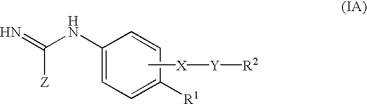

Preferably the substituent —X—Y—R² in formula (I) is in the meta position relative to the amidine group, as shown in formula (IB).

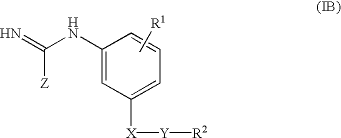

In another preferred embodiment, X represents CH₂.

In another preferred embodiment, Y represents NR³.

Preferably R1 represents C1 to 6 alkoxy. More preferably R¹ represents methoxy.

Preferably R² represents C1 to 6 alkyl-O—R¹¹. More preferably R² represents C2 alkyl-O—R¹¹. Even more preferably R² represents CH₂—CH₂—OH.

Particular compounds of the invention include:

N3{[(2R)-2(hydroxymethyl)pyrrolidinyl]methyl}-4-methoxyphenyl)-2-thiophenecarboximidamide;

N-(3-{[(2S)-2-(hydroxymethyl)pyrrolidinyl]methyl}-4-methoxyphenyl)-2-thiophenecarboximidamide;

N-(3)-{[(2-hydroxyethyl)(methyl)amino]methyl}-4-methoxyphenyl)-2-thiophenecarboximidamide,

N-[3-({[1-(hydroxymethyl)butyl]amino}methyl)-4-methoxyphenyl]-2-thiophenecarboximidamide;

N-(3-{[(2-hydroxyethyl)(methyl)amino]methyl}phenyl)-2-thilophenecarboximidamide;

N-[3-({[1-(hydroxymethyl)butyl]amino}methyl)phenyl]-2-thiophenecarboximidamide;

N-(3-{[hexyl(2-hydroxyethyl)amino}methyl]phenyl)-2-thiophenecarboximidamide;

N-(3-{[(2-hydroxyethyl)amino]methyl}-4-methoxyphenyl)-2-thiophenecarboximidamide;

N-(4-methoxy-3-{[(2-methoxyethyl)amino]methyl}phenyl-2-thiophenecarboximidamide;

N-(3-{[bis(2-hydroxyethyl)amino]methyl}-4-methoxyphenyl)-2-thiophenecarboximidamide;

N-(3-{[(cyclopropyl)(2-hydroxyethyl)amino]methyl)}-4-methoxy)-2-thiophenecarboximidamide;

N-(4-(1-ethylpropoxy)-3-{[(2-hydroxyethyl)(methyl)amino]methyl}phenyl)-2-thiophenecarboximidamide;

N-(4-(1-ethylpropoxy)-3-{[(2-hydroxyethyl)amino]methyl}phenyl)-2-thiophenecarboximidamide;

N-[3-{[(2-aminoethyl)amino]methyl}-4-(1-ethylpropoxy)phenyl]-2-thiophenecarboximidamide;

N-[4-(1-ethylpropoxy)-3-({[2-(1-piperazinyl)ethyl]amino}methyl)phenyl]-2-thiophenecarboximidamide;

N-(4-(1-ethylpropoxy)-3-{[(4-hydroxybutyl)amino]methyl}phenyl)-2-thiophenecarboximidamide;

N-(3-{[(2-hydroxyethyl)(methyl)amino]methyl}-4-isopropoxyphenyl)-2-thiophenecarboximidamide;

N-(3-{[(4-hydroxybutyl)amino]methyl}-4-isopropoxyphenyl)-2-thiophenecarboximidamide;

N-[4-isopropoxy-3-({[2-(1-piperazinyl)ethyl]amino }methyl)phenyl]-2-thiophenecarboximidamide;

N-(4-(cyclopentyloxy)-3-{[(2-hydroxyethyl)(methyl)amino]methyl}phenyl)-2-thiophenecarboximidamide;

N-(4-(cyclopentyloxy)-{[(2R)-2-(hydroxymethyl)pyrrolidinyl]methyl}phenyl)-2-thiophenecarboximidamide;

N-(3-{[(2-hydroxyethyl)(methyl)amino]methyl}-4-phenoxyphenyl)-2-thiophenecarboximidamide;

N-(3-{[(2R)-2-(hydroxymethyl)pyrrolidinyl]methyl}phenyl)-2-thiophenecarboximidamide;

N-(3-{[(2S)-2-(hydroxymethyl)pyrrolidinyl]methyl}phenyl)-2-thiophenecarboximidamide;

N-(3-{[(2-hydroxyethyl)amino]methyl}phenyl)-2-thiophenecarboximidamide;

N-(3-{[(2-methoxyethyl)amino]methyl}phenyl)-2-thiophenecarboximidamide;

N-(3-{[hexyl(2-hydroxyethyl)amino]methyl}-4-methoxyphenyl)-2-thiophenecarboximidamide;

N-(3-[3-hydroxypiperidinylmethyl]phenyl)-2-thiophenecarboximidamide;

N-(3-[4-hydroxypiperidinylmethyl]phenyl)-2-thiophenecarboximidamide;

N-(4-cyclopentyl-3-{[(2-hydroxyethyl)(methyl)amino]methyl}phenyl)-2-thiophenecarboximidamide;

N-(4-cyclopentyl-3-{[(2S)-2-(hydroxymethyl)pyrrolidinyl]methyl}phenyl)-2-thiophenecarboximidamide;

N-(4-cyclopentyl-3-{[(2R)-2-(hydroxymethyl)pyrrolidinyl]methyl}phenyl)-2-thiophenecarboximidamide;

N-(3-{[(2-hydroxyethyl)(methyl)amino]methyl}-4-methoxyphenyl)-3-thiophenecarboximidamide;

N-(3-{[(2-hydroxyethyl)amino]methyl}-2-methylphenyl)-2-thiophenecarboximnidamide;

N-(3-{[(2-hydroxyethyl)(methyl)amino]methyl}-2-methylphenyl)-2-thiophenecarboximidamide;

N-(3-{[(2R)-2-(hydroxymethyl)pyrrolidinyl]methyl}-2 -methylphenyl)-2-thiophenecarboximidarnide;

N-(5-{[(2-hydroxyethyl)(methyl)amino]ethyl}-4-methylphenyl)-2-thiophenecarboximidamide;

N-(3-{[(2-hydroxyethyl)amino]ethyl}-4-methoxyphenyl)-2-thiophenecarboximidamide;

N-(3-{-[(2-hydroxyethyl)(methyl)amino]ethyl}-4-methoxyphenyl)-2-thiophenecarboximidamide;

N-{3-[-1(2-hydroxyethyl)-2-pyrrolidinyl]-4-methoxphenyl]-2-thiophenecarboximidamide;

N-(3-(2-[benzyl(2-hydroxyethyl)amino]ethyl)phenyl)-2-thiophenecarboximidamide;

N-(3-([benzyl-(2-hydroxyethyl)amino]methyl)phenyl)-2-thiophenecarboximidamide;

N-(3-(2-[(1-hydroxymethyl)butylamino]ethyl)phenyl)-2-thiophenecarboximidamide;

and pharmaceutically acceptable salts thereof.

In one aspect the invention includes compounds of formula (I)

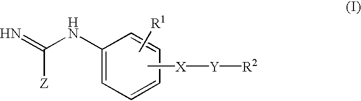

wherein

Z represents a furan or thiophene ring, optionally substituted by halogen, trifluoromethyl, C1 to 6 alkyl or C1 to 6 alkoxy;

X represents C1 to 6 alkyl;

Y represents O, S(O)ₙ or NR³;

n represents an integer 0, 1 or 2;

R¹ represents hydrogen, halogen, C1 to 6 alkyl, hydroxN, C1 to 6 alkoxy,

C1 to 6 alkoxy-O—R⁸ or C1 to 6 alkoxy-NR⁹R¹⁰;

R² represents C1 to 6 alkyl-O—R¹¹ or C1 to 6 alkyl-NR¹²R¹³;

R³ represents hydrogen, C1 to 6 alkyl or C2 to 7 alkanoyl;

or the group NR²R³ represents azetidinyl, pyrrolidinyl, piperidinyl, morpholinyl; or

piperazinyl optionally 4-substituted by C1 to 6 alkyl; each of said azacyclic rings being substituted by C1 to 6 alkyl-O—R¹⁷ or C1 to 6 alkyl-NR¹⁸R¹⁹;

R⁸, R⁹, R¹⁰, R¹¹, R¹², R¹³, R¹⁷, R¹⁸ and R¹⁹ independently represent hydrogen or C1 to6 alkyl;

or the groups NR⁹R¹⁰, NR¹²R¹³ and NR¹⁸R¹⁹ independently represent azetidinyl, pyrrolidinyl, piperidinyl, morpholinyl; or piperazinyl optionally 4-substituted by C1 to 6 alkyl;

and optical isomers, racemates and tautomers thereof and pharmaceutically acceptable salts thereof.

Unless otherwise indicated, the term “C1 to 6 alkyl” referred to herein denotes a straight or branched chain alkyl group having from 1 to 6 carbon atoms and/or a cyclic alkyl group having from 3 to 6 carbon atoms. Examples of such groups include methyl, ethyl, n-propyl, i-propyl, n-butyl, i-butyl, t-butyl, cyclopropyl, cyclopropylmethyl, cyclopentyl, methylcyclopentyl, cyclopentylmethyl and cyclohexyl.

Unless otherwise indicated, the term “C2 to 7 alkanoyl” referred to herein denotes a straight or branched chain alkyl group having from 1 to 6 carbon atoms or a cyclic alkyl group having from 3 to 6 carbon atoms bonded to a carbonyl (CO) group. Examples of such groups include acetyl, propionyl, iso-butyryl, valeryl, pivaloyl, cyclopentanoyl and cyclohexanoyl.

Unless otherwise indicated, the term “C1 to 6 alkoxy” referred to herein denotes an oxygen substituent bonded to a straight or branched chain alkyl group having from 1 to 6 carbon atoms and/or a cyclic alkyl group having from 3 to 6 carbon atoms. Examples of such groups include methoxy, ethoxy, n-propoxy, i-propoxy, n-butoxy, i-butoxy, s-butoxy, t-butoxy, cyclopropyloxy, cyclopropylmethoxy, cyclopentyloxy, methylcyclopentyloxy, cyclopentylmethoxy and cyclohexyloxy.

The term “C1 to 6 alkoxy-O—R⁸” denotes a C1 to 6 alkoxy group, as defined above, in which one hydrogen atom is replaced by a group O—R⁸.

The term “C1 to 6 alkyl-NR¹²R¹³” denotes a C1 to 6 alkyl group, as defined above, in which one hydrogen atom is replaced by a group NR¹²R¹³.

The terms “C1 to 6 alkoxy-NR⁹R¹⁰”, “C1 to 6 alkyl-O—R¹¹”, “C1 to 6 alkyl-O—R¹⁴”, “C1 to 6 alkyl-O—R¹⁷”, “C1 to 6 alkyl-NR¹⁵R¹⁶” and “C1 to 6 alkyl-NR¹⁸R¹⁹” are to be interpreted analogously.

Unless otherwise indicated, the term “halogen” referred to herein denotes fluorine, chlorine, bromine and iodine.

Examples of compounds wherein Y represents NR³ and wherein the groups X and R³ are joined together such that the group X—N—R represents a saturated 4 to 7 membered azacyclic ring include compounds such as those of formulae (IC) and (ID)

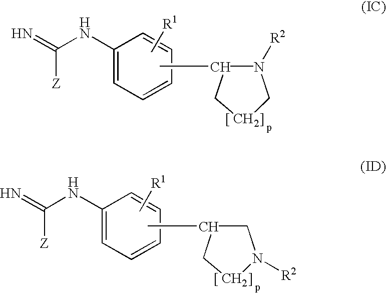

wherein p represents an integer 0 to 3.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utilits in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

Accordingy to the invention, we further provide a process for the preparation of compounds of formula (I), and optical isomers and racemates thereof and pharmaceutically acceptable salts thereof, which comprises preparing a compound of formula (I) by:

(a) reacting a corresponding compound of formula (II) or a salt thereof

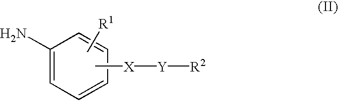

wherein R¹, R², X and Y are as defined above,

with a compound of formula (III) or a salt thereof

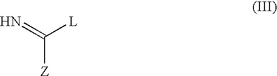

wherein Z is as defined above and L represents a leaving group; or

(b) reacting a corresponding comnpound of formula (IV) or a salt thereof

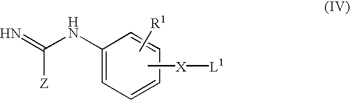

wherein R¹, X and Z are as defined above and L¹ is a leaving group, with a compound of formula H—Y—R² or a salt thereof, wherein Y and R² are as defined above; or

(c) preparing a compound of formula (b) wherein X represents —CH₂— by reduction of a corresponding compound wherein X represents —CO— (formula V)

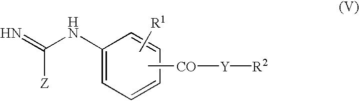

and where desired or necessary converting the resultant compound of formula (I), or another salt thereof, into a pharmaceutically acceptable salt thereof, or vice versa, and where desired converting the resultant compound of formula (I) into an optical isomer thereof.

In process (a), the reaction will take place on stirring a mixture of the reactants in a suitable solvent, for example a lower alkanol such as ethanol, 2-propanol or tert-butanol, at a temperature between room temperature and the reflux temperature of the solvent. The reaction may optionally be carried out under an atmosphere of an inert gas such as nitrogen or argon. The reaction time will depend inter alia on the solvent and the nature of the leaving group, and may be up to 48 hours; however it will rypically be from 1 to 5 hours. Suitable leaving groups L include thioalkyl, sulfonate, trifluoromethylsulfonate, halide, alkoxide, aryloxide and tosylate groups; others are recited in “Advanced Oranic Chemistry”, J. March (1985) 3^{rd} Edition on page 315 and are well known in the art. We find thioalkyl, especially thiomethyl or thioethyl, to be particularly useful.

In process (b), the displacement reaction is performed by reacting a compound of formula (IV) with a nucleophile in an inert solvent. Suitable leaving groups include sulfonate, trifluorosulfonate, tosylate, and halides selected from the group chloride, bromide or iodide. Suitable organic solvents are those such as acetonitrile, dioxane, N,N-dimethylformamide, N-methyl-2-pyrrolidinone, tetrahydrofuran, dimethylsulfoxide, sulfolane and C1 to 4 alcohols. The reaction is preferably carried out in the presence of an added base. Potential basic additives are metal carbonate, especially alkali metal carbonates, metal oxides and hydroxides, and tertiary amine bases such as diisopropylethylamine. In one preferred embodiment, the nucleophile is a primary or secondary amine and the reaction is carried out in the presence of a base. This base can be either an excess of the amine nucleophile or can be an additive to the reaction mixture. In a preferred embodiment, the leaving group is chloride.

Salts of compounds of formula (I) may be formed by reacting the free base or a salt, enantiomer, tautomer or protected derivative thereof, with one or more equivalents of the appropriate acid. The reaction may be carried out in a solvent or medium in which the salt is insoluble, or in a solvent in which the salt is soluble followed by subsequent removal of the solvent in vaczio or by freeze drying. Suitable solvents include, for example, water, dioxan, ethanol, 2-propanol, tetrahydrofuran or diethyl ether, or mixtures thereof. The reaction may be a metathetical process or it may be carried out on an ion exchange resin.

Certain novel intermediates of formulae (II), (IV) and (V) form another aspect of the invention.

Compounds of formula (II) may be prepared by methods that will be generally apparent to the man skilled in the art. In particular, these methods include the reduction of a corresponding compound of formula (VI)

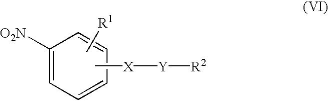

wherein R¹, R², X and Y are as defined above.

Such reductions may be achieved using various methods that are well known in the art.

Compounds of formula (III) are either known or may be prepared by known methods. For example, compounds of formula (III) in which L represents thioalkyl may be prepared by treatment of the corresponding thioamide of formula (VII)

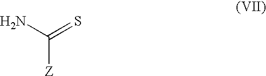

wherein Z is as defined above; with an alkyliodide.

Compounds of formula (VI) may be prepared by methods that will be generally apparent to the man skilled in the art. Such methods include:

(a) reaction of a compound of formula (VIII)

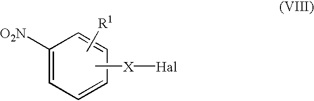

wherein R¹ and X are as defined above and Hal represents a halogen, with a nucleophile of formula (IX)

H—Y—R² (IX)

wherein R² and Y are as defined above;

(b) when X represents —CO— and R¹ represents C1 to 6 alkoxy or phenoxy, reacting a compound of formula (X)

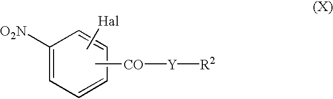

wherein R² and Y are as defined above and Hal represents a halogen, with an metal alkoxide or a metal phenoxide, M—R¹ wherein M represents a metal, particularly an alkali or alkaline earth metal such as sodium or potassium; and

(c) when Y represents NR³, by reductive amination of a compound of formula (XI)

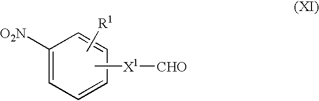

wherein X¹ represents an alkyl group having one less CH₂ group than X, and R¹ and X are as defined above; with an amine of formula (XII)

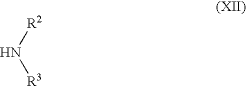

wherein R² and R³ are as defined above.

Compounds of formula (IV) may be prepared from corresponding compounds of formula (XIII)

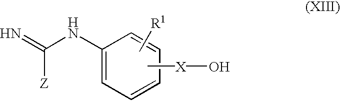

wherein R¹, X and Z are as defined above, using methods that are generally well known in the art.

Compounds of formulae (VI), (VII), (VIII), (IX), (X), (XI), (XII) and (XIII) are either known or may be prepared by conventional methods knower per se.

Intermediate compounds may be prepared as such or in protected form. In particular amine and hydroxy groups may be protected. Suitable protecting groups are described in the standard text “Protective Grouos in Organic Synthesis”, 2nd Edition (1991) by Greene and Wuts. Amine protecting groups which may be mentioned include alkyloxycarbonyl such as t-butyloxycarbonyl, phenylalkyloxycarbonyl such as benzyloxycarbonyl, or trifluoroacetate. Deprotection will normally take place on treatment with aqueous base or aqueous acid.

The compounds of the invention and intermediates may be isolated from their reaction mixtures, and if necessary further purified, by using standard techniques. The compounds of formula (I) may exist in tautomeric, enantiomeric or diastereoisomeric forms, all of which are included within the scope of the invention. The various optical isomers mav be isolated by separation of a racemic mixture of the compounds using conventional techniques, for example, fractional crystallisation or HPLC. Alternatively, the individual enantiomers may be made by reaction of the appropriate optically active starting materials under reaction conditions that will not cause racemisation.

Intermediate compounds may also exist in enantiomeric forms and may be used as purified enantiomers, diastereomers, racemates or mixtures.

The compounds of formula (I), and their pharmaceutically acceptable salts, enantiomers, racemates and tautomers, are useful because they possess pharmacological activity in animals. In particular, the compounds are active as inhibitors of the enzyme nitric oxide synthase and as such are predicted to be useful in therapy. More particularly, they are in general selective inhibitors of the neuronal isoform of the enzyme nitric oxide synthase.

The compounds and their pharmaceutically acceptable salts, enantiomers, racemates and tautomers are indicated for use in the treatment or prophylaxis of diseases or conditions in which synthesis or oversynthesis of nitric oxide synthase forms a contributory part.

Examples of such diseases or conditions include hypoxia, such as in cases of cardiac arrest, stroke and neonatal hypoxia, neurodegenerative conditions including nerve degeneration and/or nerve necrosis in disorders such as ischaemia, hypoxia, hypoglycemia, epilepsy. and in external wounds (such as spinal cord and head injury), hyperbaric oxygen convulsions and toxicity, dementia, for example, pre-senile dementia, Alzheimer's disease and AIDS-related dementia, Sydenham's chorea, Parkinson's disease, Huntington's disease, multiple sclerosis, Amyotrophic Lateral Sclerosis, Korsakoffs disease, imbecility relating to a cerebral vessel disorder, sleeping disorders, schizophrenia, anxiety, depression, seasonal affective disorder, jet-lag, depression or other symptoms associated with Premenstrual Syndrome (PMS), anxiety and septic shock.

The compounds of formula (I) are also useful in the treatment and alleviation of acute or persistent inflammatory or neuropathic pain, or pain of central orngin.

The compounds of formula (I) may also be useful in the treatment or prophylaxis of inflammation. Conditions that may be specifically mentioned include osteoartritis, rheumatoid arthritis, rheumatoid spondylitis, gouty arthritis and other arthritic conditions, inflamed joints; eczema, psoriasis, dermatitis or other inflammatory skin conditions such as sunburn; inflammatory eye conditions including uveitis and conjunctivitis; lung disorders in which inflammation is involved, for example, asthma, bronchitis, chronic obstructive pulmonary disease, pigeon fancier's disease, farmer's lung, acute respiratory distress syndrome; bacteraemia, endotoxaetnia (septic shock), aphthous ulcers, gingivitis, pyresis, pain and pancreatitis; conditions of the gastrointestinal tract including inflammatory bowel disease, Crohn's disease, atrophic gastritis, gastritis varialoforme, ulcerative colitis, coeliac disease, regional ileitis, peptic ulceration, irritable bowel syndrome, damage to the gastrointestinal tract resulting from infections by, for example, Helicobacter pylori, or from treatments with non-steroidal anti-inflammatory drugs; and other conditions associated with inflammation.

The compounds of formula (I) and their pharmaceutically acceptable salts, enantiomers, racemates and tautomers may also be useful in the treatment or prophylaxis of diseases or conditions in addition to those mentioned above. For example, the compounds may be useful in the treatment of atherosclerosis, cystic fibrosis, hvpotension associated with septic and/or toxic shock, in the treatment of dysfunction of the immune system, as an adjuvant to short-term immunosuppression in organ transplant therapy, in the treatment of vascular complications associated with diabetes and in cotherapy with cytokines, for example TNF or interleukins.

Compounds of formula (I) are also predicted to show activity in the prevention and reversal of tolerance to opiates and diazepines, treatment of drug addiction and treatment of migraine and other vascular headaches. The compounds of the present invention may also show useful immunosuppressive activity, and be useful in the treatment of gastrointestinal motility disorders, in the induction of labour, and in male contraception. The compounds may also be useful in the treatment of cancers that express nitric oxide synthase.

Compounds of formula (I) are predicted to be particularly useful in the treatment or prophylaxis of hypoxia or stroke or ischaemia or neurodegenerative conditions or schizophrenia or migraine or for the treatment of pain and especially in the treatment or prophylaxis of hypoxia or stroke or ischaemia or neurodegenerative disorders or schizophrenia or pain. We are particularly interested in the conditions selected from the group consisting of hypoxia, ischaemia, stroke, pain, anxiety, schizophrenia, Parkinson's disease, Huntington's disease, migraine and other vascular headaches.

For the treatment of Parkinson's disease, the compounds of formula (I) are expected to be particularly useful either alone, or in combination with other agents such as L-Dopa.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history ofthe disease or condition, or those who have been identified by genetic testing, or screening to be particularly susceptible to developing the disease or condition.

Thus according to a further aspect of the invention we provide a compound of formula (I), or an optical isomer or racemate thereof or a pharmaceutically acceptable salt thereof, for use as a medicament.

According to another feature of the invention we provide the use of a compound of formula (I) or an optical isomer or racemate thereof or a pharmaceutically acceptable salt thereof, in the manufacture of a medicament for the treatment or prophylaxis of the aforementioned diseases or conditions; and a method of treatment or prophylaxis of one of the aforementioned diseases or conditions which comprises administering a therapeutically effective amount of a compound of formula (I), or an optical isomer or racemate thereof or a pharmaceutically acceptable salt thereof, to a person suffering from or susceptible to such a disease or condition.

For the above mentioned therapeutic indications, the dosage administered will, of course, vary with the compound employed, the mode of administration and the treatment desired. However, in general, satisfactory results are obtained when the compounds are administered to a human at a daily dosage of between 0.5 mg and 2000 mg (measured as the active ingredient) per day, particularly at a daily dosage of between 2 mg and 500 mg.

The compounds of formula (I), and optical isomers and racemates thereof and pharmaceutically acceptable salts thereof, may be used on their own, or in the form of appropriate medicinal formulations. Administration may be by, but is not limited to, enteral (including oral, sublingual or rectal), intranasal, or topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988.

According to the invention, there is provided a pharmaceutical formulation comprising preferably less than 95% by weight and more preferably less than 50% by weight of a compound of formula (I), or an optical isomer or racemate thereof or a pharmaceutically acceptable salt thereof, in admixture with a pharmaceutically acceptable diluent or carrier.

The formulation mav optionally also contain a second pharmacologically active ingredient such as L-Dopa.

The compounds of formula (I), and pharmaceutically acceptable derivatives thereof, may also be advantageously used in combination with a COX-2 inhibitor. Particularly preferred COX-2 inhibitors are Celecoxib and MK-966. The NOS inhibitor and the COX-2 inhibitor may either be formulated together within the same pharmaceutical composition for administration in a single dosage unit, or each component may be individually formulated such that separate dosages may be administered either simultaneously or sequentially.

We also provide a method of preparation of such pharmaceutical formulations which comprises mixing the ingredients.

Examples of such diluents and carriers are: for tablets and dragees: lactose, starch, talc, stearic acid; for capsules: tartaric acid or lactose; for injectable solutions: water, alcohols, glycerin, vegetable oils; for suppositories: natural or hardened oils or waxes.

Compositions in a form suitable for oral, that is oesophageal, administration include: tablets, capsules and dragees; sustained release compositions include those in which the active ingredient is bound to an ion exchange resin which is optionally coated with a diffusion barrier to modify the release properties of the resin.

The enzyme nitric oxide synthase has a number of isoforrns and compounds of formula (I), and optical isomers and racemates thereof and pharmaceutically acceptable salts thereof, may be screened for nitric oxide synthase inhibiting activity by following procedures based on those of Bredt and Snyder in Proc. Natl. Acad. Sci., 1990, 87, 682-685. Nitric oxide synthase converts ³H-L-arginine into ³H-L-citrulline which can be separated by cation exchange chromatography and quantified by scintillation counting.

Screen For Neuronal Nitric Oxide Synthase Inhibiting Activity

The enzyme is isolated from rat hippocampus or cerebellum. The cerebellum or hippocampus of a male Sprague-Dawley rat (250-275 g) is removed following CO₂ anaesthesia of the animal and decapitation. Cerebellar or hippocampal supernatant is prepared by homogenisation in 50 mM Tris-HCl with 1 mM EDTA buffer (pH 7.2 at 25° C.) and centifugation for 15 minutes at 20,000 g. Residual L-arginine is removed from the supernatant by chromatography through Dowex AG-50W-X8 sodium form and hydrogen form columns successively, and further centrifugation at 1000 g for 30 seconds. For the assay, 25 μl of the final supernatant is added to each of 96 wells (of a 96 well filter plate) containing either 25 μl of an assay buffer (50 mM HEPES, 1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT, 100 μM NADPH, 10 μg/ml calmodulin, pH 7.4). Following a 10 minute equilibration period, 25 μl of an L-argnine solution (of concentration 18 μM ¹H-L-arginine, 96 nM ³H-L-arginine) is added to each well to initiate the reaction. The reaction is stopped after 10 minutes by addition of 200 μl of a slurry of termination buffer (20 mM HEPES, 2 mM EDTA, pH 5.5) and Dowex AG-50W-X8 200-400 mesh.

Labelled L-citrulline is separated from labelled L-arginine by filtering each filter plate and 75 μl of each terminated reaction is added to 3 ml of scintillation cocktail. The L-citrulline is then quantified by scintillation counting.

In a typical experiment using the cerebellar supernatant, basal activity is increased by 20,000 dpm/ml of sample above a reagent blank that has an activity of 7,000 dpm/ml. A reference standard, N-nitro-L-arginine, which gives 80% inhibition of nitric oxide synthase at a concentration of 1 μM, is tested in the assay to verify the procedure.

Screen For Human Neuronal Nitric Oxide Synthase Inhibiting Activity

Enzyme was isolated from human hippocampus, cortex or cerebellum. Cerebellar, cortical or hippocampal supernatant is prepared by homozerisation of frozen human tissue (1 to 5 g) in 50 mM Tris-HCl with 1 mM EDTA buffer (pH 7.2 at 25° C.) and centrifugation for 15 minutes at 20,000 g. Residual L-argiine is removed from the supernatant by chromatography through Dowex AG-50W-X8 sodium form and hydrogen form columns successively and further centrifugation at 1000 g for 30 seconds. Subsequently, the supernatant is passed through 2′-5′ ADP Sepharose and the human nNOS eluted with NADPH.

For the assay, 25 μl of the final supernatant is added to each of 96 wells (of a 96 well filter plate) containing either 25 μlof an assay buffer (50 mM HEPES, 1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT, 100 μM NADPH, 10 μg/ml calmodulin, pH 7.4). Following a 30 minute equilibration period, 25 μl of an L-arginine solution (of concentration 12 μM ¹H-L-arginine, 96 nM ³H-L-arginine) is added to each test tube to initiate the reaction. The reaction is stopped after 30 minutes by addition of 200 μl of a slurry of termination buffer (20 mM HEPES, 2 mM EDTA, pH 5.5) and Dowex AG-50W-XS 200-400 mesh.

Labelled L-citrulline is separated from labelled L-arginine by filtering each filter plate and 75 μl of each terminated reaction is added to 3 ml of scintillation cocktail. The L-citrulline is then quantified by scintillation counting.

In a typical experiment using the cerebellar supernatant, basal activity is increased by 20,000 dpm/ml of sample above a reagent blank that has an activity of 7,000 dpm/ml. A reference standard, N-nitro-L-arginine, which gives 80% inhibition of nitric oxide synthase at a concentration of 1 μM, is tested in the assav to verifv the procedure.

Screen For Human Inducible Nitric Oxide Synthase Inhibiting Activity

Partially purified iNOS was prepared from cultured and yvsed human DLD1 cells which had been activated with TNF-alpha, interferon gamma, and LPS. Centrifugation at 1000 g, removed cellular debris and residual L-arginine was removed from the supernatant by chromatography through Dowex AG-50W-X8 sodium form and hydrogen form columns successively.

For the assay, 25 μl of the final supernatant is added to each of 96 wells (of a 96 well filter plate) containing either 25 μl of an assay buffer (50 mM HEPES, 1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT, 100 μM NADPH, 10 μg/ml calmodulin, pH 7.4). Following a 30 minute equilibration period, 25 μl of an L-arginine solution (of concentration 12 μM ¹H-L-arginine, 96 nM ³H-L-arginine) is added to each test tube to initiate the reaction. The reaction is stopped after 30 minutes by addition of 200 μl of a slurry of termination buffer (20 mM HEPES, 2 mM EDTA, pH 5.5) and Dowex AG-50W-X8 200-400 mesh.

Labelled L-citrulline is separated from labelled L-argzinine by filtering each filter plate and 75 μl of each terminated reaction is added to 3 ml of scintillation cocktail. The L-citrulline is then quantified by scintillation counting.

In a typical experiment using the DLD1 supernatant, basal activity is increased by 10,000 dpm/ml of sample above a reagent blank that has an activity of 5,000 dpm/ml. A reference standard, N-methyl-L-arginine, which gives 80% inhibition of nitric oxide synthase at a concentration of 1 μM, is tested in the assay to verify the procedure.

Screen For Endothelial Nitric Oxide Synthase Inhibiting Activity

The enzyme is isolated from human umbilical vein endothelial cells (HUVECs) by a procedure based on that of Pollock et al in Proc. Natl. Acad. Sci., 1991, 88, 10480-10484. HUVECs were purchased from Clonetics Corp (San Diego, Calif., USA) and cultured to confluency. Cells can be maintained to passage 35-40 without significant loss of yield of nitric oxide synthase. When cells reach confluency, they are resuspended in Dulbecco's phosphate buffered saline, centrifuged at 800 rpm for 10 minutes, and the cell pellet is then homogenised in ice-cold 50 mM Tris-HCl, 1 mM EDTA, 10% glycerol, 1 mM phenylmethylsulphonylfluoride, 2 μM leupeptin at pH 4.2. Following centrifuaation at 34,000 rpm for 60 minutes, the pellet is solubilised in the homogenisation buffer which also contains 20 mM CHAPS. After a 30 minute incubation on ice, the suspension is centrifuged at 34,000 rpm for 30 minutes. The resulting superiatant is stored at −80° C. until use.

For the assay, 25 μl of the final supernatant is added to each of 12 test tubes containin 25 μl L-arginine solution (of concentration 12 μM ¹H-L-aroinine, 64 nM ³H-L-arginine) and either 25 μl of an assay buffer (50 mM HEPES, 1 nmM EDTA, 1.5 mM CaCl₂, pH 7.4) or 25 μl of test compound in the buffer at 22° C. To each test tube was added 25 μl of complete assay buffer (50 mM HEPES, 1 mM EDTA, 1.5 mM CaCl₂, 1 mM DTT, 100 μM NADPH, 10 μg/ml calmodulin, 12 μM tetrahydrobiopterin. pH 7.4) to initiate the reaction and the reaction is stopped after 10 minutes by addition of 2 ml of a termination buffer (20 mM HEPES, 2 mM EDTA, pH 5.5).

Labelled L-citrulline is separated from labelled L-arginine by chromatography over a Dowex AG-50W-X8 200-400 mesh column. A 1 ml portion of each terminated reaction mixture is added to an individual 1 ml column and the eluant combined with that from two 1 ml distilled water washes and 16 ml of scintillation cocktail. The L-citrulline is then quantified by scintillation counting.

In a typical experiment, basal activity is increased by 5,000 dpm/ml of sample above a reagent blank that has an activity of 1500 dpm/ml. A reference standard, N-nitro-L-arginine, which gives 70-90% inhibition of nitric oxide synthetase at a concentration of 1 μM, is tested in the assay to verify the procedure.

In the screens for nitric oxide synthase inhibition activity, compound activity is expressed as IC₅₀ (the concentration of drug substance which gives 50% enzyme inhibition in the assay). IC₅₀ values for test compounds were initially estimated from the inhibiting activity of 1, 10 and 100 μM solutions of the compounds. Compounds that inhibited the enzyme by at least 50% at 10 μM were re-tested using more appropriate concentrations so that an IC₅₀ could be determined.

When tested in the above screens, the compounds of Examples 1 to 43 below show IC₅₀ values for inhibition of neuronal nitric oxide synthase of less than 10 μM and good selectivity compared to inhibition of the endothelial isoform of the enzyme, indicating that they are predicted to show particularly useful therapeutic activity.

The invention is illustrated but in no way limited by the following examples:

### PREPARATION 1

2-Thiophenecarboximidothioic Acid Ethyl Ester Hydrochloride

To a stirred solution of ethanethiol (28.4 g, 450 mmol) in dichloromethane (500 mL) at 10° C. under nitrogen was added 2-thiophenecarbonitrile (50.0 g, 450 mmol). The solution was treated with a slow stream of hydrogen chloride gas for 6 h. The reaction mixture was then allowed to warm to room temperature. After 18 h diethyl ether (200 mL) was added and a white solid crystallized out. The solid 2-thiophenecarboximidothioic acid ethyl ester hydrochloride was collected by filtration and air dried (65.8 g, 83%); m.p. 196-197° C.

### PREPARATION 2

2-Furancarboximidothioic Acid Ethyl Ester Hydrochloride

Following the procedure described in Preparation 1 but substituting 2-furancarbonitrile for 2-thiophenecarbonitrile, the title compound was prepared as a white solid in 23% yield; MS: ^{m}/z 156 [M+H]⁺.

### PREPARATION 3

2-Methoxy-5-Nitrobenzaldehyde

To triphenylphosphine (11.9 g, 45 mmol) in tetrahydrofuran (100 ml) was added 2-hydroxy-5-nitrobenzaldehyde (6.3 g, 38 mmol) and methanol (1.8 g, 57 mmol) followed by diethyl azodicarboxylate (7.9 g, 45 mmol) and the reaction mixture was let stir at room temperature for 1 h. The solvent was evaporated off, the residue was dissolved in ethyl acetate (50 mL) and filtered through a plug of silica. The filtrate was concentrated and chromatographed on silica gel using a gradient of 20-50% ethyl acetate in methanol to afford the title compound (4.9 g, 71.6%) as an oil; MS: ^{m}/z 182 [M+H]⁺.

### PREPARATION 4

2-Methoxy-3-Nitrobenzaldehyde

Following the procedure described in Preparation 3 but subsituting 2-hydroxy-3-nitrobenzaldehyde for 2-hydroxy-5-nitrobenzaldehyde, the title compound was obtained as a yellow oil in 54% yield; MS: ^{m}/z 182 [M+H]⁺.

### PREPARATION 5

4-Methoxy-3-Nitrobenzaldehyde

Following the procedure described in Preparation 3 but substituting 4-hydroxy-3-nitrobenzaldehyde for 2-hydroxy-5-nitrobenzaldehyde, the title compound was obtained as a yellow oil in 40% yield; MS: ^{m}/z 182 [M+H]⁺.

### PREPARATION 6

2-Chloro N-Methyl-5-Nitrobenzamide

To a solution of 2-chloro-5-nitrobenzoyl chloride (20 g, 91 mmol) in dichloromethane (200 mL) cooled to 0° C. was added an ice-cold solution of dichloromethane (100 mL) containing methylamine (11 g, 360 mmol). The reaction mixture was stirred in the cold for 1 h, diluted with dichloromethane (500 mL), and the dichloromethane solution washed sequentially with water (200 mL), 5% aqueous hydrochloric acid (150 mL), water (200 mL) and aqueous saturated sodium chloride solution (2×150 mL). The dichloromethane solution was then dried over magnesium sulphate and the solvent evaporated to afford the title compound (15.5 g, 80%) as a colourless solid; MS: ^{m}/z215 [M+H]⁺.

### PREPARATION 7

N-[3-(Chloromethyl)-4-Methoxyphenyl]-2-Thiophenecarboximidamide Hydrochloride

a) Methyl 2-Methoxy-5-Nitrobenzoate

To methyl 2-methoxybenzoate (5.0 g, 30 mmol) dissolved in cold (0° C.) sulphuric acid (25 mL) was added portionwise potassium nitrate (3.0 g, 30 mmol) and the mixture was allowed to stir at 0° C. for 1 h. The reaction mixture was poured onto 200 mL of ice/water mixture and the solid was collected, washed well with water and air-dried; yield 3.5 g (58%); MS: ^{m}/z 212 [M+H]⁺.

b) Methyl 5-Amino-2-Methoxybenzoate Hydrochloride

To a Parr pressure bottle charged with methyl 2-methoxy-5-nitrobenzoate (3.5 g, 17.4 mmol) in ethanol (200 mL) was added an ethanol solution saturated with hydrogen chloride (20 mL) followed by 10% Pd/C (200 mg) and the mixture was hydrogenated at 45 psi for 1 h. The catalyst was filtered off and the solvent evaporated to afford the title compound (3.6 g, 100%) as a colourless solid; MS: ^{m}/z 182 [M+H]⁺.

c) Methyl 5-{Imino(2-Thienyl)Methyl]amino}-2-Methoxybenzoate

To methyl 5-amino-2-methoxybenzoate hydrochloride (45.6 g, 210 mmol) dissolved in ethanol (250 mL) was added pyridine (16.7 g, 210 mmol) followed by 2-thiophenecarboximidothioic acid ethyl ester hydrochloride (50.2 g, 240 mmol) and the reaction mixture was heated at 60° C. under nitrogen for 18 h. The reaction mixture was poured into water (2 L) and the solid which formed was collected, washed with ether (1 L) and air-dried; yield 41.2 g (67%), m.p. 154-155° C.

d) N-[3-(Hydroxymethyl)-4-Methoxyphenyl]-2-Thiophenecarboximidamide

To a stirred suspension of lithium aluminium hydride (9.8 g, 260 mmol) in dry tetrahydrofuran (40 mL) at 0° C. under nitrogen was added dropwise a solution containing methyl 5-{[imino(2-thienyl)methyl]amino}-2-methoxybenzoate (38.2 g, 130 mmol) in tetrahydrofuran (100 mL) and the mixture was stirred for a further 2 h at 0° C. To the cooled solution was then added water (10 mL), followed by 15% aqueous ammonia (10 mL) and finally water (10 mL). The aluminium salts were filtered off and the solvent evaporated to afford a light yellow solid (24 g). The material was slurried in ether and the product collected; yield of nearly colourless solid (23.4 g, 68.8%); m.p. 191-192° C.

e) N-[3-(Chloromethyl)-4-Methoxyphenyl]-2-Thiophenecarboximidamide Hydrochloride

To a stirred solution containing N-[3-(hydroxymethyl)-4-methoxyphenyl]-2-thiophenecarboximidamide (14.2 g, 54 mmol) dissolved in dichloromethane (200 mL) was added dropwise thionyl chloride (19.3 g, 160 mmnol) and the mixture was stirred at room temperature for 2 h. The mixture was poured into diethyl ether (1.5 L), stirred for 16 h and the precipitate collected, washed well with diethyl ether and air-dried; yield 15.4 g (97%), m.p. 204-205° C.

### PREPARATION 8

N-[3-(Chloromethyl)-4-(1-Ethylpropoxy)Phenyl]-2-Thiophenecarboximidamide Hydrochloride

a) 2-(1-Ethylpropoxy)-5-Nitrobenzoic Acid

To sodium hydride (4.5 g, 60% in oil; 106 mmol) suspended in DMSQ (100 mL) was added 2-chloro-5-nitrobenzoic acid (10 g, 48 mmol) followed by 3-pentanol (6 mL, 55 mmol) and the reaction mixture was stirred at 60° C. under nitrogen for 5 days. The mixture was allowed to cool to room temperature, made acidic by the dropwise addition of 2N hydrochloric acid and extracted with ethyl acetate (3×100 mL). The organic extracts were combined, dried over magnesium sulphate and evaporated to afford an oil (15 g). The oil was chromatographed on silica gel using chloroform/methanol, 95:5, as eluent to afford the title compound (10.4 g, 85%) as an off-white solid; MS: ^{m}/z 254 [M+H]⁻.

b) Methyl 2-(1-Ethylpropoxy)-5-Nitrobenzoate

To a solution containing 2-(1-ethylpropoxy)-5-nitrobenzoic acid (10.4 g, 41 mmol) in anhydrous methanol (200 mL) was added thionyl chloride (3.7 mL, 51 mmol) dropwise with stirring. The resulting solution was heated at reflux for 3 h, cooled to room temperature and the solvent evaporated to afford the title compound (11 g, 100%) as a light yellow oil; MS: ^{m}/z 268 [M+H]⁺.

c) Methyl 5-Amino-2-(1-Ethylpropoxy)Benzoate

To a solution containing methyl 2-(1-ethylpropoxy)-5-nitrobenzoate (11 g, 41 mmol) in 95% ethanol (150 mL) was added 10% Pd/C (100 mg) and the mixture was hydrogenated in a Parr apparatus at an initial pressure of 45 psi for 1 h. The catalyst was filtered off and the filtrate concentrated to give a dark oil (9.2 g). The oil was chromatographed on silica gel using ethyl acetate/hexane, 2 3, as eluent to afford the title compound (7.7 g, 80%) as a colourless oil; MS: ^{m}/z 238 [M+H]⁻.

d) Methyl 2-(1-Ethylpropoxy)-5-{[Imino(2-Thienyl)Methyl]amino}Benzoate

Following the same procedure as used in the preparation of intermediate 7(c) above but substituting methyl 5-amino-2-(1-ethylpropoxy)benzoate for methyl 5-amino-2-methoxybenzoate hydrochloride the title compound was prepared in 90% yield; MS: ^{m}/z 347 [M+H]⁺.

e) N-[4-(1-Ethylpropoxy)-3-(Hydroxymethyl)Phenyl]-2-Thiophenecarboximidamide

Following the same procedure as used in the preparation of intermediate 7(d) above but substituting methyl 2-(1-ethylpropoxy)-5- {[imino(2-thienyl)methyl]amino}benzoate for methyl 5-{imino(2-thienyl)methyl]amino}-2-methoxybenzoate the title compound was prepared in 92% yield; MS: ^{m}/z 319 [M+H]⁺.

f) N-[3-(Chloromethyl)-4-(1-Ethylpropoxy)Phenyl]-2-Thiophenecarboximidamide Hydrochloride

Following the same procedure as used in the preparation of intermediate 7(e) above but substituting N-[4-(1-ethylpropoxy)-3-(hydroxymethyl)phenyl]-2-thiophenecarboximidamide for N-[3-(hydroxymethyl)-4-methoxyphenyl]-2-thiophenecarboximidamide the title compound was prepared in 90% yield; MS: ^{m}/z 337 [M+H]⁺.

### PREPARATION 9

N-[3-(Chloromethyl)-4-Isopropoxyphenyl]-2-Thiophenecarboximidamide Hydrochloride

a) 2-Isopropoxy-5-Nitrobenzoic Acid

Following the same procedure as used in the preparation of intermediate 8(a) above but substituting 2-propanol for 3-pentanol the title compound was prepared in 70% yield; MS :^{m}/z226 [M+H]⁺.

b) Methyl 2-Isopropoxy-5-Nitrobenzoate

Following the same procedure as used in the preparation of intermediate 8(b) above but substituting 2-isopropoxy-5-nitrobenzoic acid for 2-(1-ethylpropoxy)-5-nitrobenzoic acid the title compound was obtained as a yellow solid in 93% yield; MS: ^{m}/z 240 [M+H]⁺.

c) Methyl 5-Amino-2-Isopropoxybenzoate

Following the same procedure as used in the preparation of intermediate 8(c) above but substituting methyl 2-isopropoxy-5-nitrobenzoate for methyl 2-(1-ethylpropoxy)-5-nitrobenzoate the title compound was prepared in quantitative yield; MS: ^{m}/z 210 [M+H]⁺.

d) Methyl 2-Isopropoxy-5-{[Imino(2-Thienyl)Methyl]Amino}Benzoate

Following the same procedure as used in the preparation of intermediate 7(c) above but substituting methyl 5-amino-2-isopropoxybenzoate for methyl 5-amino-2-methoxybenzoate hydrochloride the title compound was prepared in 93% Yield; MS: ^{m}/z 319[M+H]⁺.

e) N-[3-(Hydroxymethyl)-4-Isopropoxyphenyl]-2-Thiophenecarboximidamide

Following the same procedure as used in the preparation of intermediate 7(d) above but substituting methyl 2-isopropoxy-5-{[imino(2-thienyl)methyl]amino}benzoate for methyl 5-{imino(2-thienyl)methyl]amino}-2-methoxybenzoate the title compound was obtained as an off-white solid in 95% yield; MS: ^{m}/z 291 [M+H]⁺.

f) N-[3-(Chloromethyl)-4-Isopropoxyphenyl]-2-Thiophenecarboximidamide Hydrochloride

Following the same procedure as used in the preparation of intermediate 7(e) above but substituting N-[3-(hydroxymethyl)-4-isopropoxyphenyl]-2-thiophenecarboximidamide for N-[3-(hydroxymethyl)-4-methoxyphenyl]-2-thiophenecarboximidamide the title compound was prepared in 90% yield; MS: ^{m}/z 309 [M+H]⁺.

### PREPARATION 10

N-[3-(Chloromethyl)-4-(Cyclopentyloxy)Phenyl]-2-Thioohenecarboximidamide Hydrochloride

a) 2-Cyclopentyloxy-5-Nitrobenzoic Acid

Following the same procedure as used in the preparation of intermediate 8(a) above but substituting cyclopentanol for 3-pentanol the title compound was prepared in 82% yield; MS: ^{m}/z 252 [M+H]⁺.

b) Methyl 2-Cyclopentyloxy-5-Nitrobenzoate

Following the same procedure as used in the preparation of intermediate 8(b) above but substituting 2-cyclopentyloxy-5-nitrobenzoic acid for 2-(1-ethylpropoxy)-5-nitrobenzoic acid the title compound was prepared in 95% yield; MS: ^{m}/z 266 [M+H]⁺.

c) Methyl 5-Amino-2-Cyclopentyloxybenzoate

Following the same procedure as used in the preparation of intermediate 8(c) above but substituting methyl 2-cyclopentyloxy-5-nitrobenzoate for methyl 2-(1-ethylpropoxy)-5-nitrobenzoate the title compound was prepared in quantitative yield; MS: ^{m}/z 236 [M+H]⁺.

d) Methyl 2-Cyclopentyloxy-5-{[Imino(2-Thienyl)Methyl]Amino}Benzoate

Following the same procedure as used in the preparation of intermediate 7(c) above but substituting methyl 5-amino-2-cyclopentyloxybenzoate for methyl 5-amino-2-methoxybenzoate hydrochloride the title compound was prepared in 88% yield; MS: ^{m}/z 345 [M+H]⁺.

e) N-[4-Cyclopentyloxy-3-(Hydroxymethyl)Phenyl]-2-Thiophenecarboximidamide

Following the same procedure as used in the preparation of intermediate 7(d) above but substituting methyl 2-cyclopentyloxy-5-{[imino(2-thienyl)methyl]amino}benzoate for methyl 5-{imino(2-thienyl)methyl]amino}-2-methoxybenzoate the title compound was prepared in 80% yield; MS: ^{m}/z 317 [M+H]⁺.

f) N-[3-(Chloromethyl)-4-Cyclopentyloxyphenyl]-2-Thiophenecarboximidaride Hydrochloride

Following the same procedure as used in the preparation of intermediate 7(e) above but substituting N-[4-cyclopentyloxy-3-(hydroxymethyl)phenyl]-2-thiophenecarboximidamide for N-[3-(hydroxymethyl)-4-methoxyphenyl]-2-thiophenecarboximidamide the title compound was prepared in 30% yield; MS: ^{m}/z 335 [M+H]⁺.

### PREPARATION 11

N-[3-(Chloromethyl)-4-Phenoxyphenyl]-2-Thiophenecarboximidamide Hydrochloride

a) 5-Nitro-2-Phenoxybenzoic Acid

Following, the same procedure as used in the preparation of intermediate 8(a) above but substituting phenol for 3-pentanol the title compound was obtained as a light brown solid in 78% yield; MS: ^{m}/z 260 [M+H]⁺.

b) Methyl 5-Nitro-2-Phenoxybenzoate

Following the same procedure as used in the preparation of intermediate 8(b) above but substituting 5-nitro-2-phenoxybenzoic acid for 2-(1-ethylpropoxy)-5-nitrobenzoic acid the title compound was obtained as a light tan solid in 96% yield; MS: ^{m/z} 274 [M+H]⁺.

c) Methyl 5-Amino-2-Phenoxybenzoate

Following the same procedure as used in the preparation of intermediate 8(c) above but substituting methyl 5-nitro2-phenoxybenzoate for methyl 2-(1-ethylpropoxy)-5-nitrobenzoate the title compound was obtained as a colourless solid in quantitative yield; MS: ^{m}/z 244 [M+H]⁺.

d) Methyl 5-{[Imino(2-Thienyl)Methyl]amino}-2-Phenoxybenzoate

Following the same procedure as used in the preparation of intermediate 7(c) above but substituting methyl 5-amino-2-phenoxybenzoate for methyl 5-amino-2-methoxybenzoate hydrochloride the title compound was obtained as a cream-coloured solid in 78% yield; MS: ^{m}/z 353 [M+H]⁺.

e) N-[3-(Hydroxymethyl)-4-Phenoxyphenyl]-2-Thiophenecarboximidamide

Following the same procedure as used in the preparation of intermediate 7(d) above but substituting methyl 5-{[imino(2-thienyl)methyl]amino}-2-phenoxybenzoate for methyl 5-{}imino(2-thienyl)methyl]amino}-2-methoxybenzoate the title compound was obtained as a light yellow solid in 80% yield; MS: ^{m}/z 325 [M+H]⁻.

f) N-[3-(Chloromethyl)-4-Phenoxyphenyl]-2-Thiophenecarboximidamide Hydrochloride

Following the same procedure as used in the preparation of intermediate 7(e) above but substituting N-[3-(hydroxymethyl)-4-phenoxyphenyl]-2-thiophenecarboximidamide for N-[3 -(hydroxymethyl)-4-methoxyphenyl]-2-thiophenecarboximidamide the title compound was obtained as a tan solid in 55% yield; MS: ^{m}/z: 343 [M+H]⁺.

### PREPARATION 12

N-[5-(Chloromethyl)-2-Methoxyphenyl]-2-Thiophenecarboximridamide Hydrochloride

a) Methyl 4-Methoxy-3-Nitro-Benzoate

Following the same procedure as used in the preparation of intermediate 8(b) above but substituting 4-methoxy-3-nitrobenzoic acid for 2-(1-ethylpropoxy)-5-nitrobenzoic acid the title compound was prepared in 81% yield; MS: ^{m}/z 212 [M+H]⁺.

b) Methyl 3-Amino-4-Methoxybenzoate

Following the same procedure as used in the preparation of itermediate 8(c) above but substituting methyl 4-methoxy-3-nitrobenzoate for methyl 2-(1-ethylpropoxy)-5-nitrobenzoate the title compound was prepared in quantitative yield; MS: ^{m}/z 182 [M+H]⁺.

c) Methyl 3-{[Imino(2-Thienyl)Methyl]amino}-4-Methoxybenzoate

Following the same procedure as used in the preparation of intermediate 7(c) above but substituting methyl 3-amino-4-methoxybenzoate for methyl 5-amino-2-methoxybenzoate hydrochloride the title compound was prepared in 33% yield; MS: ^{m}/z 291 [M+H]⁻.

d) N-[5-(Hydroxymethyl)-2-Methoxyphenyl]-2-Thiophenecarboximidamide

Following the same procedure as used in the preparation of intermediate 7(d) above but substituting methyl 3-{[imino(2-thienyl)methyl]amino}-4-methoxybenzoate for methyl 5-{imino(2-thienyl)methyl]amino}-2-methoxybenzoate the title compound was prepared in 97% yield; MS: ^{m}/z 263 [M+H]⁺.

e) N-[5-(Chloromethyl)-2-Methoxyphenyl]-2-Thiophenecarboximidamide Hydrochloride

Following the same procedure as used in the preparation of intermediate 7(e) above but substituting N-[5-(hydroxymethyl)-2-methoxyphenyl]-2-thiophenecarboximidamide for N-[3-(hydroxymethyl)-4-methoxyphenyl]-2-thiophenecarboximidamide the title compound was prepared in 90% yield; MS: ^{m}/z 281 [M+H]⁺.

### PREPARATION 13

N-[3-(Chloromethyl)-4-Cyclopentylphenyl]-2-Thiophenecarboximidamide Hydrochloride

a) Methyl 2-Cyclopentylbenzoate

To a solution of methyl 2-(2-cyclopenten-1-yl)benzoate (1.2 g), prepared by the method described in Tetrahedron Lett., 1988, 29, 905-908, dissolved in ethanol (250 mL) was added 10% Pd/C (100 mg) and the mixture was hydrogenated in a Parr apparatus at an initial pressure of 45 psi until the theoretical uptake of hydrogen was complete. The catalyst was filtered off and the filtrate concentrated to afford the title compound (1.1 g) as a colourless oil; MS: ^{m}/z 205 [M+H]⁺.

b) Methyl 2-Cyclopentyl-5-Nitrobenzoate

To an ice-cold solution containing methyl 2-cyclopentylbenzoate (1.1 g, 5.4 mmol) dissolved in trifluoroacetic acid (1.6 mL) was added nitric acid (90%, 0.25 mL, 5.6 mmol) followed by the dropwise addition of trifluoroacetic anhydride (1 mL). The reaction mixture was allowed to warm to room temperature and then stir at room temperature for a further 3 h. The reaction was made basic with aqueous ammonia and extracted with ethyl acetate (2×20 mL). The organic extracts were combined, dried (magnesium sulphate) and evaporated to afford a yellow oil (1.3 g). Chromatography on silica gel using hexane/ethyl acetate, 9:1, as eluent afforded the title compound (1.0 g, 77%) as an amber oil; MS: ^{m}/z 250 [M+H]⁺.

c) Methyl 5-Amino-2-Cyclopentylbenzoate

Following the same procedure as used in the preparation of intermediate 8(c) above but substituting methyl 2-cyclopentyl-5-nitrobenzoate for methyl 2-(1-ethylpropoxy)-5-nitrobenzoate the title compound was prepared in quantitative yield; MS: ^{m}/z 220 [M+H]⁺.

d) Methyl 2-Cyclopentyl-5-{[Imino(2-Thienyl)Methyl]Amino}Benzoate

Following the same procedure as used in the preparation of intermediate 7(c) above but substituting methyl 5-amino-2-cyclopentylbenzoate for methyl 5-amino-2-methoxybenzoate hydrochloride the title compound was prepared in 66% yield; MS: ^{m}/z 329 [M+H]⁺.

e) N-[4-Cyclopentyl-3-(Hydroxymethyl)Phenyl]-2-Thiophenecarboximidamide

Following the same procedure as used in the preparation of intermediate 7(d) above but substituting methyl 2-cyclopentyl-5-{[imino(2-thienyl)methyl]amino}benzoate for methyl 5-{imino(2-thienyl)methyl]amino}-2-methoxybenzoate the title compound was prepared in 97% yield; MS: ^{m}/z 301 [M+H]⁺.

f) N-[3-(Chloromethyl)-4-Cyclopentylphenyl]-2-Thiophenecarboximidamide Hydrochloride

Following the same procedure as used in the preparation of intermediate 7(e) above but substituting N-[4-cyclopentyl-3-(hydroxymethyl)phenyl]-2 -thiophenecarboximidamide for N-[3 -(hydroxymethyl)-4-methoxyphenyl]-2-thiophenecarboximidamide the title compound was prepared in 95% yield; MS: ^{m}/z 319 [M+H]⁺.

### PREPARATION 14

3-Thiophenecarboximidothioic Acid Ethyl Ester Hydrochloride

Following the procedure described in the preparation of intermediate 1 but substituting 3-thiophenecarbonitrile for 2-thiophenecarbonitrile the title compound was prepared as a white solid in 35% yield; MS: ^{m}/z 172 [M+H]⁺.

### PREPARATION 15

N-(3-Chloromethyl-2-Methylphenyl)Thiophene-2-Carboxamidine

a) (3-Amino-2-Methylphenyl)Methanol

To a stirred suspension containing 3-amino-2-methylbenzoic acid (9.09 g, 60.1 mmol) in of tetrahydrofuran (225 mL) at 0° C. was added 1M borare-tetrahydrofuran complex (120 mL) dropwise. The suspension was stirred for 30 minutes at 0° C. and then refluxed for 2.5 h at 75° C. After 1 h, a large white mass formed and had to be broken up so that the reaction could continue stirring. The suspension was treated with methanol dropwise to slowly quench the reaction and then water (25 mL) was added. The tetrahydrofuran was removed under reduced pressure and the residue was taken up in chloroform and washed with 1N sodium hydroxide solution. The aqueous phase was extracted with chloroform (4×100 mL) and the combined organic layers were then dried (sodium sulphate), filtered and concentrated under reduced pressure to afford the title compound (6.59 g, 80%) as a white solid; MS: ^{m}/z 138 [M+H]⁺.

b) N-[3-Hydroxymethyl-2-Methylphenyl)Thiophene-2-Carboxamidine

To a stirred solution containing (3-amino-2-methylphenyl)methanol (6.01 g, 43.8 mmol) in ethanol (55 mL) was added 2-thiophenecarboximidothioic acid ethyl ester hydrochloride (11.8 g, 54.8 mmol) and the solution was heated to 60° C. for 6 h. A gas scrubber with bleach was attached in order to neutralise the ethanethiol released during the reaction. The solution was treated with water (50 mL) and then basified to pH 11 with aqueous ammonium hydroxide. The aqueous layer was extracted with ethyl acetate (3×20 mL) and the combined organic layers were dried (sodium sulphate), filtered and concentrated under reduced pressure to give an orange oil/solid. The crude product was triturated with diethyl ether (4×20 mL) to afford the title compound (7.76 g, 72%) as a white solid; MS: ^{m}/z245 [M+H]⁺.

c) N-[3-Chloromethyl-2-Methylphenyl)Thiophene-2-Carboxamidine Hydrochloride

To a stirred suspension containing N-(3-hydroxymethyl-2-methylphenyl)thiophene-2-carboxamidine (5.95 g, 24.1 mmol) in dichloromethane (85 mL) was added thionyl chloride (5.28 mL, 72.4 mmol) dropwise. The N-(3-hydroxymethyl-2-methylphenyl)thiophene-2-carboxamidine went into solution as the thionyl chloride was added. The solution stirred for 2 h at ambient temperature then diethyl ether (50 mL) was added and stirred until the product finished precipitating out of solution. The organic solvents were removed under reduced pressure to afford the title compound (7.51 g, 92%) as a tan solid; MS: ^{m}/z 265 [M+H]⁺.

### PREPARATION 16

N-(3-Acetyl-4-Methoxyphenyl)-2-Thiophenecarboximidamide

(a) N-(3-Acetyl-4-Methoxyphenyl)Butanamide

N-(3-Acetyl-4-hydroxyphenyl)butanamide (20.9 g, 95 mmol) and potassium carbonate (42.5 g, 308 mmol) were stirred in DMF (200 ml) under nitrogen. lodomethane (12 ml, 27.3 g, 192 mmol) was added, and stirring was continued ovemight. The solution was evaporated, and the residue was partitioned between ethyl acetate and water. The organic layer was dried (magnesium sulfate), filtered, and recrystallised from ethanol to give the sub-title compound (16.9 g, 72 mmol, 76%) as a colourless solid. m.p. 113° C.; MS (ES⁻) ^{m}/z 236 (100%, MH⁻).

(b) 1-(5-Amino-2-Methoxyphenyl)Ethanone

N-(3-Acetyl-4-methoxyphenyl)butanamide (3.00 g, 12.8 mmol) was dissolved in a 1:1 mixture of concentrated hydrochloric acid and water. The solution was stirred at 100° C. for 1 h. The solution was allowed to cool, then basified with aqueous sodium hydroxide and extracted with dichloromethane. The organic layer was dried (magnesium sulfate), filtered, and evaporated to give the sub-title compound as an oil (1.96 g, 11.9 mmol, 93%). A sample of the compound was dissolved in methanol, excess hydrocen chloride (4M in dioxane) was added, then the solution was evaporated giving the hydrochloride salt of the sub-title compound as a solid. MS (ES⁻) ^{m}/z 166 (MH⁻).

(c) N-(3-Acetyl-4-Methoxyphenyl)-2-Thiophenecarboximidamide

A solution containing 1-(5-amino-2-methoxyphenyl)ethanone (1.54 g, 9.3 mmol) and 2-thiophenecarboximidothioic acid ethyl ester hydrochloride (2.24 g, 10.8 mmol) in ethanol (30 ml) was heated at 60° C. under nitrogen overnight. The solution was evaporated. The residue was stirred with aqueous potassium carbonate for about 1 h. The resulting solid was collected by filtration, washed with water, then dried under vacuum. Recrystallisation from ethyl acetate/hexane gave the title compound as a pale solid (1.92 g, 7.00 mmol, 75%). MS (ES⁻) ^{m}/z 275 (100%, MH⁻).

### PREPARATION 17

1(5-Bromo-2-Methoxyphenyl)-4-Chloro-1-Butanone

A mixture of 4-chlorobutanoyl chloride (25 ml, 31.45 g, 250 mmol) and 4-bromoanisole (25 ml, 37.35 g, 200 mmol) was added dropwise to a solution of aluminium chloride in nitrobenzene (1M, 250 ml, 250 mmol) which was stirred at 0° C. under nitrogen. Stirring was continued overnight and the solution was allowed to warm slowly to room temperature. The solution was poured into ice, and the resulting solution was then evaporated under vacuum. The residue was dissolved in ether, and the solution was filtered. Rotary evaporation of the ether followed by Kugelrohr distillation of the residue (280° C., oven temperature) gave the title compound as an oil. MS (ES⁺) ^{m}/z 290, 292, 294 (MH⁺).

### PREPARATION 18

N-(2-Hydroxyethyl)-5-{[Imino(2-Thienyl)Methyl]Amino}-2-Methoxybenzamide Hydrochloride

a) 2-Chloro-N-(Hydroxyethyl)-5-Nitrobenzamide

To an ice-cold solution of 2-chloro-5-nitrobenzoyl chloride (5.0 g, 22.7 mmol) dissolved in dichloromethane (20 mL) was added ethanolamine (1.4 g, 22.7 mmol) followed immediately by saturated aqueous sodium hydrogen carbonate (3 mL). After 1 h. the solvent was removed, the residue slurried with water, filtered and dried to afford the title compound in 75% yield; MS: ^{m}/z 245 [M+H]⁺.

b) N-(Hydroxyethyl)-2-Methoxy-5-Nitrobenzamide

To a solution of sodium methoxide in methanol, prepared by dissolving sodium metal (0.63 g, 27 mmol) in methanol (25 mL), was added, in rapid drops, a solution of 2-chloro-N-(hydroxyethyl)-5-nitrobenzamide (4.1 g, 16 mmol) in methanol (25 mL) and the reaction mixture was heated at 50° C. for 18 h. The mixture was cooled, the solvent evaporated and the yellow-orange solid residue dissolved in chloroform (150 mL). The chloroform solution was washed with water (2×50 mL), dried over magnesium sulphate, filtered and evaporated to afford the title compound (1.4 g, 35%) as a yellow solid; MS: ^{m}/z 241 [M+H]⁺.

c) 5-Amino-N-(Hydroxyethyl)-2-Methoxybenzamide

Following the procedure described in Example 1(b) the nitro compound obtained in Preparation 8(b) was reduced to the title compound in 95% yield; MS: ^{m}/z 211 [M+H]⁺.

d) N-(2-Hydroxyethyl)-5-{[Imino(2-Thienyl)Methyl]Amino}-2-Methoxybenzamide Hydrochloride

Using the method described in Example 1(c) but substituting 5-amino-N-(hydroxyethyl)-2-methoxybenzamide for [(2R)-1-(5-amino-2-methoxybenzyl)-pyrrolidinyl]methanol hydrochloride, the title compound was obtained as a colourless solid in 10% yield; MS: ^{m}/z 320 [M+H]⁺.

### EXAMPLE 1

N-(3-{[(2R)-2-(Hydroxymethyl)Pyrrolidinyl]methyl}-4-Methoxyphenyl)-2-Thiophenecarboximidamide Dihydrochloride

a) [(2R)-1-(2-Methoxy-5-Nitrobenzyl)Pyrrolidinyl]Methanol Hydrochloride

To 2-methoxy-5-nitrobenzyl bromide (6.11 g, 24.8 mmol) dissolved in N,N-dimethylformamide (25 mL) was added Hunig's base (3.5 g, 27.2 mmol) followed by R-(−)-2-pyrrolidinemethanol (2.4 g, 24.8 mmol) and the reaction mixture was stirred at room temperature for 16 h. The N,N-dimethylformamide was removed, the residue treated with water (100 mL) followed by aqueous ammonium hydroxide until basic and extracted with ethyl acetate (3×100 mL). The ethyl acetate extracts were combined, washed with water (3×100 mL), dried over magnesium sulphate, filtered, and evaporated to give the free base of the title compound as a yellow oil (6.0 g). Treatment of an ethanol solution of the free base with ethanol/hydrogen chloride afforded the title compound (7.4 g, 95%) as a light yellow solid; MS: ^{m}/z 267 [M+H]⁺.

b) [(2R)-1-(5-Amino-2-Methoxybenzyl)Pyrrolidinyl]Methanol Hydrochloride

To [(2R)-1-(2-methoxxy-5-nitrobenzyl)pyrrolidinyl]methanol hydrochloride (7.4 g) in 95% ethanol (300 mL) was added a catalytic amount (˜100 mg) of 10% Pd/C and the mixture was hydrogenated at 45 psi until the required amount of hydrogen was taken up (approximately 2 h). The catalyst was filtered off and the solvent evaporated to afford the title compound (6.5 g, 97%); MS: ^{m}/z 237 [M+H]⁺.

c) N-(3-{[(2R)-2-(Hydroxymethyl)Pyrrolidinyl]methyl}-4-Methoxyphenyl)-2-Thiophenecarboximidamide Dihydrochloride

To [(2R)-1-(5-amino-2-methoxybenzyl)pyrrolidinyl]methanol hydrochloride (6.5 g, 23.7 mmol) in ethanol (50 mL) was added 2-thiophenecarboximidothioic acid ethyl ester hydrochloride (5.9 g, 28.2 mmol) and the solution was stirred under an atmosphere of nitrogen with heating to 60° C. for 6 h. Water (75 mL) was then added, the solution basified to pH 11 with aqueous aimmonium hydroxide, and the aqueous solution extracted with ethyl acetate (3×250 mL). The ethyl acetate extracts were combined, dried over magnesium sulphate and evaporated to afford a viscous brown oil (4.2 g). Chromatography on silica gel using chloroform:methanol, 8:2, afforded the free base of the title compound as a light tan solid. Dissolution in 2-propanol and acidification with diethyl ether/hydrogen chloride afforded the title compound as a tan solid in 10% yield; MS: ^{m}/z 346 [M+H]⁺.

### EXAMPLE 2

N-(3-{[(2S)-2-(Hydroxymethyl)Pyrrolidinyl]methyl}-4-Methoxyphenyl)-2-Thiophenecarboximidamide Dihydrochloride

a) [(2S)-1-(2-Methoxy-5-Nitrobenzyl)Pyrrolidinyl]Methanol Hydrochloride

Following the same procedure as in Example 1(a) but substituting S-(+)-2-pyrrolidinemethanol for R-(−)-2-pyrrolidinemethanol, the title compound was prepared in 93% yield; MS: ^{m}/z 267 [M+H]⁺.

b) [(2S)-1-(5-Amino-2-Methoxybenzyl)Pyrrolidinyl]Methanol Hydrochloride

Following the procedure described in Example 1(b) the nitro compound obtained in Example 2(a) was reduced to the title compound in 95% yield; MS: ^{m}/z 237 [M+H]⁺.

c) N-(3-{[(2S)-2-(Hydroxymethyl)Pyrrolidinyl]Methyl}-4-Methoxyphenyl)-2-Thiophenecarboximidamide Dihydrochloride

Using the method described in Example 1(c) but substituting [(2S)-1-(5-amino-2-methoxybenzyl)pyrrolidinyl]methanol hydrochloride for [(2R)-1-(5-amino-9-methoxybenzyl)pyrrolidinyl]methanol hydrochloride, the title compound was obtained as a tan solid in 25% yield; MS: ^{m}/z 346 [M+H]⁺.

### EXAMPLE 3

N-(3-{[(2-Hydroxyethyl)(Methyl)Amino]Methyl}-4-Methoxyphenyl)-2-Thiophenecarboximidamide Dihydrochloride

a) 2-[(2-Methoxy-5-Nitrobenzyl)(Methyl)Amino]Ethanol Hydrochloride

Following the same procedure as in Example 1(a) but substituting 2-(methylamino)ethanol for R-(−)-2-pyrrolidinemethanol, the title compound was prepared in 70.7% yield; MS: ^{m}/z 241 [M+H]⁺.

b) 2-[(5-Amino-2-Methoxybenzyl)(Methyl)Amino]Ethanol Hydrochloride

Following the procedure described in Example 1(b) the nitro compound obtained in Example 3(a) was reduced to the title compound in 95% ,ield; MS: ^{m}/z 211 [M+H]⁺.

c) N-(3-{[(2-Hydroxyethyl)(Methyl)Amino]Methyl}-4-Methoxyphenyl)-2-Thiothenecarboximidamide Dihydrochloride

Using the method described in Example 1(c) but substituting 2-[(5-amino-2-methoxybenzyl)(methyl)amino]ethanol hydrochloride for [(2R)-1-(5-amino-2-methoxybenzyl)pyrrolidinyl]methanol hydrochloride, the title compound was obtained as a colourless solid in 66% yield; MS: ^{m}/z 320 [M+H]⁺.

### EXAMPLE 4

N-[3-{[1-(Hydroxymethyl)Butyl]Amino}Methyl)-4-Methoxylhenyl]-2-Thiophenecarboximidamide Ditrifluoroacetate

a) 2-[(2-Methoxy-5-Nitrobenzyl)amino]-1-Pentanol Hydrochloride

Following the same procedure as in Example 1(a) above but substituting 2-amino-1-pentanol for R-(−)-2-pyTrolidinemethanol, the title compound was prepared in 90% yield; MS: ^{m}/z 269 [M+H]⁺.

b) 2-[(5-Amino-2-Methoxybenzyl)Amino]-1-Pentanol Hydrochloride

Following the procedure described in Example 1(b) the nitro compound obtained in Example 4(a) was reduced to the title compound in 95% yield; MS: ^{m}/z 239 [M+H]⁺.

c) N-[3-{[1-(Hydroxymethyl)Butyl]Amino}Methyl)-4-Methoxyphenyl]-2-Thiothenecarboximidamide Ditrifluoroacetate

Using the method described in Example 1(c) but substituting 2-[(5-amino-2-methoxybenzyl)amino]-1-pentanol hydrochloride for [(2R)-1-(5-amino-2-methoxybenzyl)pyrrolidinyl]methanol hydrochloride, the title compound was obtained, after conversion of the free base into the ditnifluoroacetate salt, as a colourless solid in 35% yield; MS: ^{m}/z 343 [M+H]⁺.

### EXAMPLE 5

N-(3-{[(2-Hydroxyethyl)(Methyl)Amino]Methyl}Phenyl)-2-Thiophenecarboximidamide Dihydrochloride

a) 2-[(3-Nitrobenzyl)(Methyl)Amino]Ethanol Hydrochloride

To 3-nitrobenzylchloride (5.0 g, 29 mmol) dissolved in N,N-dimethylformamide (100 mL) was added triethylamine (4.3 g, 44 mmol) followed by 2-(methylamino)ethanol (2.2 g, 29 mmol) and the reaction mixture was stirred at room temperature for 16 h. The N,N-dimethylformamide was removed, the residue treated with water (100 mL) followved by aqueous ammonium hydroxide until basic and extracted with ethyl acetate (3×100 mL). The ethyl acetate extracts were combined, washed with water (3×100 mL), dried over magnesium sulphate, filtered, and evaporated to give the free base of the title compound as a yellow oil (6.1 g). Treatment of an ethanol solution of the free base with ethanol/hydrogen chloride afforded the title compound (5.8 g, 81%) as a light yellow solid; MS: ^{m}/z 211 [M+H]⁺.

b) 2-[(3-Aminobenzyl)(Methyl)Amino]Ethanol Hydrochloride

Following the procedure described in Example 1(b) the nitro compound obtained in Example 5(a) was reduced to the title compound in 95% yield; MS: ^{m}/z 181 [M+H]⁺.

c) N-(3-{[(2-Hydroxyethyl)(Methyl)Amino]Methyl}Phenyl)-2-Thiophenecarboximidamide Dihydrochloride

Using the method described in Example 1(c) but substituting 2-[(3-aminobenzyl)-(methyl)amino]ethanol hydrochloride for [(2R)-1-(5-amino-2-methoxybenzyl)-pyrrolidinyl]methanol hydrochloride, the title compound was obtained as a colourless solid in 36% yield; MS: ^{m}/z 290 [M+H]⁺.

### EXAMPLE 6

N-[3-{[1-(Hydroxymethyl)Butyl]Amino}Methyl)Phenyl]-2-Thiophenecarboximidamide Ditrifluoroacetate

a) 2-[(3-Nitrobenzyl)Amino]Pentanol

Following the same procedure as in Example 5(a) but substituting 2-amino-1-pentanol for 2-(methylamino)ethanol, the title compound was prepared in 92% yield; MS: ^{m}/z 239 [M+H]⁺.

b) 2-[(3-Aminobenzyl)Amino]-1-Pentanol Hydrochloride

Following the procedure described in Example 1(b) the nitro compound obtained in Example 6(a) was reduced to the title compound in 95% yield: MS: ^{m}/z 209 [M+H]⁺.

c) N-[3-{[1-(Hydroxymethyl)Butyl]Amino}Methyl]Phenyl]-2-Thiophenecarboximidamide Ditrifluoroacetate

Using the method described in Example 1(c) but substituting 2-[(3-aminobenzyl)amino]-1-pentanol hydrochloride for [(2R)-1-(5-amino-2-methoxybenzyl)-pyrrolidinyl]methanol hydrochloride, the title compound was obtained, after conversion of the free base into the ditrifluoroacetate salt, as a colourless solid in 12% yield; MS: ^{m}/z 318 [M+H]⁺.

### EXAMPLE 7

N-(3-{[Hexyl(2-Hydroxyethyl)Amino}Methyl]Phenyl)-2-Thiophenecarboximidamide Ditrifluoroacetate

a) 2-[Hexyl(3-Nitrobenzyl)Amino]Ethanol

To a solution of 2-(hexylamino)ethanol (2.9 g, 20 mmol) in acetonitrile (20 mL) was added potassium carbonate (8.0 g, 58 mmol) followed by 3-nitrobenzylchloride (5.0 g, 20 mmol) and the mixture was stirred at room temperature under an atmosphere of nitrogen for 16 h. The solid was filtered off, the solvent evaporated and the residue chromatographed on silica gel using ethyl acetate/hexane to afford the title compound (5.1 g, 63%) as an oil; MS: ^{m}/z 281 [M+H]⁺.

b) 2-[(3-Aminobenyl)(Hexyl)Amino]Ethanol

Following the procedure described in Example 1(b) the nitro compound obtained in Example 7(a) was reduced to the title compound in 95% yield; MS: ^{m}/z 251 [M+H]⁺.

c) N-(3-{[Hexyl(2-Hydroxyethyl)Amino}Methyl]Phenyl)-2-Thiophenecarboximidamide Ditrifluoroacetate

Using the method described in Example 1(c) but substituting 2-[(3-aminobenzyl) (hexyl)amino]ethanol for [(2R)-1-(5-amino-2-methoxbenzyl)-pyrrolidinyl]methanol hydrochloride, the title compound was obtained, after conversion of the free base into the ditrifluoroacetate salt, as a colourless solid in 20% yield; MS: ^{m}/z 360 [M+H]⁺.

### EXAMPLE 8

N-(3-{[(2-Hydroxyethyl)Amino]Methyl}-4-Methoxyphenyl)-2-Thiophenecarboximidamide Dihydrochloride

a) 2-[(2-Methoxy-5-Nitrobenzyl)Amino]Ethanol Hydrochloride

Following the same procedure as in Example 1(a) but substituting 2-aminoethanol for R-(−)-2-pyrrolidinemethanol, the title compound was prepared in 85% yield; MS: ^{m}/z 227 [M+H]⁺.

b) 2-[(5-Amino-2-Methoxybenzyl)Amino]Ethanol Hydrochloride

Following the procedure described in Example 1(b) the nitro compound obtained in Example 8(a) was reduced to the title compound in 95% yield; MS: ^{m}/z 197 [M+H]⁺.

c) N-(3-{[(2-Hydroxyethyl)Amino]Methyl}-4-Methoxyohenyl)-2-Thiophenecarboximidamide Dihydrochloride

Using the method described in Example 1(c) but substituting 2-[(5-amnino-2-methoxybenzyl)amino]ethanol hydrochloride for [(2R)-1-(5-amino-2-methoxybenzyl)pyrrolidinyl]methanol hydrochloride, the title compound was obtained as a colourless solid in 15% yield; MS: ^{m}/z 306 [M+H]⁺.

### EXAMPLE 9

N-(4-Methoxy-3-{[(2-Methoxyethyl)Amino]Methyl}Phenyl-2-Thiophenecarboximidamide Hydrochloride

a) 2-Methoxy-N-(2-Methoxy-5-Nitrobenzyl)Ethanamine

Following the same procedure as in Example 1(a) but substituting 2-methoxyethylamine for R-(−)-2-pyrrolidinemethanol, the title compound was prepared in 85% yield, MS: ^{m}/z 241 [M+H]⁺.

b) 4-Methoxy-3-{[(2-Methoxyethyl)Amino]Methyl}Aniline Hydrochloride

Following the procedure described in Example 1(b) the nitro compound obtained in Example 9(a) was reduced to the title compound in 95% yield; MS: ^{m}/z 211 [M+H]⁺.

c) N-(4-Methoxy-3-{[(2 -Methoxyethyl)Amino]Methyl}Phenyl-2-Thiophenecarboximidamide Hydrochloride

Using the method described in Example 1(c) but substituting 4-methoxy-3-{[(2-methoxyethyl)amino]methyl}aniline hydrochloride for [(2R)-1-(5-amino-2-methoxybenzyl)pyrrolidinyl]methanol hydrochloride, the title compound was obtained as a colourless solid in 25% yield; MS: ^{m}/z 320 [M+H]⁺.

### EXAMPLE 10

N-(3-{[Bis(2-Hydroxyethyl)Amino]Methyl}-4-Methoxyphenyl)-2-Thiophenecarboximidamide Dihydrochloride

a) 2-[(2-Methoxy-5-Nitrobenzyl)Amino]Bisethanol Hydrochloride

Following, the same procedure as in Example 1(a) but substituting diethanolamine for R-(−)-2-pyrrolidinemethanol the title compound was prepared in 56% yield; MS: ^{m}/z 271 [M+H]⁺.

b) 3-{[Bis(2-Hydroxyethyl)Amino]Methyl}-4-Methoxyanilmne

To 2-[(2-methoxy-5-nitrobenzyl)amino]bisethanol hydrochloride (1.8 g, 5.9 mmol) dissolved in methanol (20 mL) and acetic acid (2 mL) was added portionwise zinc dust (6.65 g, 100 mmol). The reaction mixture was heated at refiux for 16 h., cooled to room temperature and the remaining unreacted zinc was filtered off. The methanol was removed, the residue taken up in water (100 mL) / chloroform (100 mL), basified with aqueous ammonia and the mixture allowed to stir for 1 h. The zinc salts were filtered off, the chloroform layer separated, washed with brine, dried (magnesium sulphate) and evaporated to afford the title compound (0.9 g, 64%) as an oil; MS: ^{m}/z 241 [M+H]⁺.

c) N-(3-{[Bis(2-Hydroxyethyl)Amino]Methyl}-4-Methoxyphenyl)-7-Thiophenecarboximidamide Dihydrochloride

Using the method described in Example 1(c) but substiruting 3-{[bis(2-hydroxyethyl)amino]methyl}-4-methoxyaniline for [(2R)-1-(5-amino-2-methoxybenzyl)pyrrolidinyl]methanol hydrochloride the title compound was obtained as a colourless solid in 52% yield; MS: ^{m}/z 350 [M+H]⁺.

### EXAMPLE 11

N-(3-{[(Cyclopropyl)(2-Hydroxyethyl)Amino]Methyl}-4-Methoxy)-2-Thiophenecarboximidamide

a) Methyl [Cyclotropyl(2-Methoxy-5-Nitrobenzyl)Amino]Acetate

A mixture of 2-methoxy-5-nitrobenzaldehyde (1.97 g), methyl (cyclopropylamino)acetate hydrochloride (2 g) in 1% acetic acid/methanol (40 ml) was stirred at room temperature for 30 minutes. Sodium cyanoborohydride (1.35 g) was added and reaction was stirred for 3.5 h. The reaction mixture was diluted with water and solid sodium carbonate was added, theh the mixture was extracted with dichloromethane and the extracts were dried (magnesium sulfate), filtered and evaporated. The compound was purified by flash chromatography using 2%-5% methanol in dichloromethane to give the sub-title compound as a light yellow oil (2.22 g); MS: ^{m}/z 395 [M+H]⁺.

b) Methyl [(5-Amino-2-Methoxybenzyl)(Cyclopropyl)Amino]Acetate

To the methyl [cyclopropyl(2-methoxy-5-nitrobenzyl)amino]acetate in ethanol (50 ml) was added 10% Pd-C (200 mg). The mixture was hydrogenated at 50 psi for 2.5 h. The catalyst was filtered through Celite. The solution was evaporated to give the sub-title compound (1.85 g) which was used without further purification; MS: ^{m}/z 365 [M+H]⁺.

c) Methyl [Cyclopropyl(5-{[Imino(2-Thienyl)Methyl]Aminol]-2-Methoxybenzyl)Amino}acetate

To methyl [(5-amino-2-methoxybenzyl)(cyclopropyl)amino]acetate (1.85 g) in ethanol (5 ml) was added 2-thiophenecarboximidothioic acid ethyl ester hydrochloride (2.18 g) and the solution was refixed for 4.5 h. The reaction mixture was diluted with water and solid potassium carbonate was added. The aqueous layer was extracted with chloroform. The extract was dried (magnesium sulfate), filtered and evaporated. The compound was purified by reverse phase HPLC on a Waters Bondapak® C₁₈ column usin a gradient of acetonitrile and 0.1% aqueous trifluoroacetic acid as the eluent. The free base product was prepared by basification of the product containing fractions with potassium carbonate. The precipitate was filtered, washed with water and dried under vacuum to give the title compound as a white solid; MS: ^{m}/z 374 [M+H]⁺.

d) N-(3-{[(Cyclopropyl)(2-Hydroxyethyl)Amino]Methyl}-4-Methoxy)-2-Thiophenecarboximidamide

To the solution of methyl [cyclopropyl(5-{[imino(2-thienyl)methyl]amino}-2-methyoxybenzyl)amino]acetate (536 mg) in tetrahydrofuran (20 ml) was added lithium aluminium hydride (1.7 ml, 1M in tetrahydrofuran) at 0° C. The mixture was stirred at 0° C. for 1 h and at room temperature for 2 days. A few drops of water were added and the solid was filtered. The compound was purified by reverse phase HPLC on a Waters Bondapak® C₁₈ column using a gradient of acetonitrile and 0.1% aqueous trifluoroacetic acid as the eluent. The free base product was prepared by basification of the product-containing fractions with potassium carbonate filtration of the precipitate which was then dried under vacuum to give the title compound as a colourless solid (226 mg); MS: ^{m}/z 346 [M+H]⁺.

### EXAMPLE 12

N-(4-(1-Ethylpropoxy)-3-{[(2-Hydroxyethyl)(Methyl)Amino]Methyl}Phenyl)-2-Thiophenecarboximidamide Dihydrochloride

To N-[3-(chloromethyl)-4-(1-ethylpropoxy)phenyl)]-2-thiophenecarboximidamide hydrochloride (0.52 g, 1.4 =mol) dissolved in DMF (20 mL) was added diisopropylethylamine (1.2 mL, 7.0 mmol) followed by 2-(methylamino)ethanol (0.56 mL, 7.0 mmol) and the reaction mixture was stirred at room temperature for 16 h. The mixture was diluted with water. Excess potassium carbonate was added. The aqueous layer was extracted with dichloromethane then dried (magnesium sulfate), filtered, and evaporated. The residue was purified by reverse phase HPLC on a Waters Bondapak® C₁₈ column using a gradient of acetonitrile and 0.1% aqueous trifluoroacetic acid as the eluent. The free base was prepared by basification of the product-containing fractions with potassium carbonate and extraction with dichloromethane. The organic extract was dried (magnesium sulfate), filtered, and evaporated. Dissolution of the residual oil from evaporation of the extracts in methanol, addition of excess hydrogen chloride solution (1M in diethyl ether) and evaporation gave the title compound as a light yellow solid (234 mg; 36%); MS: ^{m}/z 376 [M+H]⁺.

### EXAMPLE 13

N-(4-(1-Ethylpropoxy)-3-{[(2-Hydroxyethyl)Amino]Methyl}Phenyl)-2-Thiophenecarboximidamide Dihydrochloride

Using the method described in Example 12 but substituting 2-aminoethanol for 2-(methylamino)ethanol the title compound was obtained as a colourless solid in 30% yield; MS: ^{m}/z 362 [M+H]⁺.

### EXAMPLE 14

N-[3-{[(2-Aminoethyl)Amino]Methyl}-4-(1-Ethylpropoxy)Phenyl]-2-Thiophenecarboximidamide Trihydrochloride

Using the method described in Example 12 but substituting ethylene diamine for 2-(methylamino)ethanol the title compound was obtained as a colourless solid in 31% yield; MS: ^{m}/z 361 [M+H]⁺.

### EXAMPLE 15

N-[4-(1-Ethylpropoxy)-3-({[2-(1-Piperazinyl)Ethyl]Amino}Methyl)Phenyl]-2-Thiophenecarboximidamide Tetrahydrochloride

Using the method described in Example 12 but substituting 2-(1-piperazinyl)ethanamine for 2-(methylamino)ethanol the title compound was obtained as a colourless solid in 24% yield; MS: ^{m}/z 430 [M+H]⁺.

### EXAMPLE 16

N-(4-(1-Ethylproloxy)-3-{[(4-Hydroxybutyl)Amino]Methyl}Phenyl)-2-Thiophenecarboximidamide Dihydrochloride

Using the method described in Example 12 but substituting 4-amino-1-butanol for 2-(methylamino)ethanol the title compound was obtained as a colourless solid in 32% yield; MS: ^{m}/z 390 [M+H]⁺.

### EXAMPLE 17

N-(3-{[(2-Hydroxyethyl)(Methyl)Amino]Methyl}-4-Isopropoxyphenyl)-2-Thiophenecarboximidamide

Prepared by a method analogous to that described in Example 12 from N-[3-(chloromethyl)-4-isopropoxyphenyl]-2-thiophenecarboximidamide hydrochloride, 2-(methylamino)ethanol and diisopropylethylamine in DMF. The solution was diluted with water. Potassium carbonate was added and the precipitate was collected by filtration, then washed with water, and dried under vacuum to give the title compound as a yellow solid; MS: ^{m}/z 348 [M+H]⁺.

### EXAMPLE 18

N-(3-{[(4-Hydroxybutyl)Amino]Methyl}-4-Isopronoxyohenyl)-2-Thiophenecarboximidamide Dihydrochloride

Using the method described in Example 12 substituting 4-amino-1-butanol for 2-(methylamino)etahnol and substituting N-[3-(chloromethyl)-4-isopropoxyphenyl]-2-thiophenecarboximidamide hydrochloride for N-[3-(chloromethyl)-4-(1-ethylpropoxy)phenyl)]-2-thiophenecarboximidamide hydrochloride the title compound was obtained as a colourless solid in 17% yield; MS: ^{m}/z 362 [M+H]⁺.

### EXAMPLE 19

N-[4-Isopropoxy-3-({[2-(1-Piperazinyl)Ethyl]Amino}Methyl)Phenyl]-2-Thiophenecarboximidamide Trihydrochloride

Using the method described in Example 12 but substituting 2-(1-piperazinyl)ethanamine for 2-(methylamino)ethanol and substituting N-[3-(chloromethyl)-4-isoprpoxyphenyl]-2-thiophenecarboximidamide hydrochloride for N-[3-(chloromethyl)-4-(1-ethylpropoxy)phenyl)]-2-thiophenecarboximidamide hydrochloride the title compound was obtained as a colourless solid in 33% yield; MS: ^{m}/z 402 [M+H]⁺.

### EXAMPLE 20

N-(4-(Cyclopentyloxy)-3-{[(2-Hydroxyethyl)(Methyl)Amino]Methyl)}Phenyl)-2-Thiophenecarboximidamide

Using the method described in Example 12 but substituting N-[3-(chloromethyl)-4-cyclopentyloxyphenyl]-2-thiophenecarboximidamide hydrochloride for N-[3 -(chloromethyl)-4-(1-ethylpropoxy)phenyl)]-2-thiophenecarboximidamide hydrochloride the title compound was obtained (as a free base) as a colourless solid in 66% yield; MS: ^{m}/z 374 [M+H]⁺.

### EXAMPLE 21

N-(4-(Cyclopentyloxy)-3{[(2R)-2-(Hydroxymethyl)Pyrrolidinyl]Methyl}Phenyl)-2-Thiophenecarboximidamide

Using the method described in Example 12 but substituting (R)-(−)-2-pyrrolidinemethanol for 2-(methylamino)ethanol and substituting N-[3-(chloromethyl)-4-cyclopentyloxyphenyl]-2-thiophenecarboximidamide hydrochloride for N-[3-(chloromethyl)-4-(1-ethylpropoxy)phenyl)]-2-thiophenecarboximidamide hydrochloride the title compound was obtained (as a free base) as a colourless solid in 30% yield; m.p. 126-127° C.; MS: ^{m}/z 400 [M+H]⁺.

### EXAMPLE 22

N-(3-{[(2-Hydroxyethyl)(Methyl)Amino]Methyl}-4-Phenoxyphenyl)-2-Thiophenecarboximidamide Hydrochloride

Using the method described in Example 12 but substituting N-[3-(chloromethyl)-4-phenoxyphenyl]-2-thiophenecarboximidamide hydrochloride for N-[3-(chloromethyl)-4-(1-ethylpropoxy)phenyl)]-2-thiophenecarboximidamide hydrochloride the title compound was obtained as a colourless solid in 25% yield; MS: ^{m}/z 382 [M+H]⁺.

### EXAMPLE 23

N-(3-{[(2R)-2-(Hydroxymethyl)Pyrrolidinyl]Methyl}Phenyl)-2-Thiophenecarboximidamide Dihydrochloride

a) [(2R)-1-(3-Nitrobenzyl)Pyrrolidinyl]Methanol Hydrochloride

Following the same procedure in Example 5(a) but substituting R-(−)-2-pyrrolidinemethanol for 2-(methylamino)ethanol the title compound was prepared in 95% yield; MS: ^{m}/z 237 [M+H]⁺.

b) [(2R)-1-(3-Aminobenzyl)Pyrrolidinyl]Methanol Hydrochloride

Following the procedure described in Example 1(b) the nitro compound of Example 23(a) was reduced to the title compound in 95% yield; MS: ^{m}/z 207 [M+H]⁺.

c) N-(3-{[(2R)-2-(Hydroxymethyl)Pyrrolidinyl]Methyl}Phenyl)-2-Thiophenecarboximidatnide Dihydrochloride

Using the method described in Example 1(c) but substituting [(2R)-1-(3-aminobenzyl)pyrrolidinyl]methanol hydrochloride for [(2R)-1-(5-amino-2-methoxybenzyl)-pyrrolidinyl]methanol hydrochloride the title compound was obtained as a colourless solid in 30% yield; MS: ^{m}/z 316 [M+H]⁺.

### EXAMPLE 24

N-(3-{[(2S)-2-(Hydroxymethyyl)Pyrrolidinyl]Methyl}Phenyl)-2-Thiophenecarboximidamide Dihydrochloride

a) [(2S)-1-(3-Nitrobenzyl)Pyrrolidinyl]Methanol Hydrochloride

Following the same procedure as in Example 5(a) but substituting S-(−)-2-pyrrolidinemethanol for 2-(methylamino)ethanol the title compound was prepared in 87% yield; MS: ^{m}/z 237 [M+H]⁺.

b) [(9S)-1-(3-Aminobenzyl)Pyrrolidinyl]Methanol Hydrochloride

Following the procedure described in Example 1(b) the nitro compound in Example 24(a) was reduced to the title compound in 93% yield; MS: ^{m}/z 207 [M+H]⁺.

c) N-(3-{[(2S)-2-(Hydroxymethyl)Pyrrolidinyl]Methyl}Phenyl)-2-Thiophenecarboximidamide Dihydrochloride

Using the method described in Example 1(c) but substituting [(2S)-1-(3-aminobenzyl)pyrrolidinyl]methanol hydrochloride for [(2R)-1-(5-amino-2-methoxybenzyl)-pyrrolidinyl]methanol hydrochloride the title compound was obtained as a colourless solid in 25% yield; MS: ^{m}/z 316 [M+H]⁺.

### EXAMPLE 25

N-(3-{[(2-Hydroxyethyl)Amino]Methyl}Phenyl)-2-Thiophenecarboximidamide Dihydrochloride

a) 2-[(3-Nitrobenzyl)Amino]Ethanol Hydrochloride

Following the procedure of Example 5(a) but substituting ethanolamine for 2-(methylamino)ethanol the title compound was prepared in 77% yield; MS: ^{m}/z 197 [M+H]⁺.

b) 2-[(3-Aminobenzyl)Amino]Ethanol Hydrochloride

Following the procedure described in Example 1(b) the nitro compound of Example 25(a) was reduced to the title compound in quantitative yield; MS: ^{m}/z 167 [M+H]⁺.

c) N-(3-{[(2-Hydroxyethyl)Amino]Methyl}Phenyl)-2-Thiophenecarboximidamide Dihydrochloride

Using the method described in Example 1(c) but substituting 2-[(3-aminobenzyl)amino]ethanol hydrochloride for [(2R)-1-(5-amino-2-methoxybenzyl)-pyrrolidinyl]methanol hydrochloride the title compound was obtained as a colouriess solid in 35% yield; MS: ^{m}/z 277 [M+H]⁺.

### EXAMPLE 26

N-(3-{[(2-Methoxyethyl)Amino]Methyl}Phenyl)-2-Thiophenecarboximidamide Dihydrochloride

a) 2-Methoxy-N-(3-Nitrobenzyl)Ethanamine Hydrochloride

Following the procedure of Example 5(a) but substituting 2-methoxy ethylamine for 2-(methylamino)ethanol the title compound was prepared in 93% yield; MS: ^{m}/z 211 [M+H]⁺.

b) 3-{[2-Methoxyethyl)Amino]Methyl}Aniline Hydrochloride

Following the procedure described in Example 1(b) the nitro compound of Example 26(a) was reduced to the title compound in quantitative yield; MS: ^{m}/z 181 [M+H]⁺.

c) N-(3-{[(2-Methoxyethyl)Amino]Methyl}Phenyl)-2-Thiophenecarboximidamide Dihydrochloride

Using the method described in Example 1(c) but substituting 3-{[(2-methoxyethyl)amino]methyl}aniline hydrochloride for [(2R)-1-(5-amino-2-methoxybenzyl)-pyrrolidinyl]methanol hydrochloride the title compound was obtained as a colourless solid in 32% yield; MS: ^{m}/z 290 [M+H]⁺.

### EXAMPLE 27

N-(3-{[Hexyl(2-Hydroxyethyl)Amino]Methyl}-4-Methoxphenyl)-2-Thiophenecarboximidamide Dihydrochloride

a) 2-[Hexyl(2-Methoxy-5-Nitrobenzyl)Amino]Ethanol Hydrochloride

Following the procedure of Example 1(a) but substituting 2-(hexylamino)ethanol for R-(−)-2-pyrrolidinemethanol the title compound was prepared in 75% yield; MS: ^{m}/z 311 [M+H]⁺.

b) 2-[(5-Amino-2-Methoxybenzyl)(Hexyl)Amino]Ethanol Hydrochloride

Following the procedure described in Example 1(b) the nitro compound of Example 27(a) was reduced to the title compound in 95% yield; MS: ^{m}/z 281 [M+H]⁺.

c) N-(3-{[Hexyl(2-Hydroxyethyl)Amino]Methyl}-4-Methoxyphenyl)-2-Thiophenecarboximidamnide Dihydrochloride

Using the method described in Example 1(c) but substiruting 2-[(5-amino-2-methoxybenzyl)(hexyl)amino]ethanol hydrochloride for [(2R)-1-(5-amino-2-methoxybenzyl)pyrrolidinyl]methanol hydrochloride the title compound was obtained as a colourless solid in 40% yield; MS: ^{m}/z 391 [M+H]⁺.

### EXAMPLE 28

N-(3-[3 -Hydroxypiperidinylmethyl]phenyl)-2-Thiophenecarboximidamide Dihydrochloride

MS: ^{m}/z 316 [M+H]⁺.

### EXAMPLE 29

N-(3-[4-Hydroxypiperidinylmethyl]Phenyl)-2-Thiophenecarboximidamide Dihydrochloride

MS: ^{m}/z 316 [M+H]⁺.

### EXAMPLE 30

N-(4-Cyclopentyl-3-{[(2-Hydroxyethyl)(Methyl)Amino]Methyl}Phenyl)-2-Thiophenecarboximidamide

Using the method described in Example 12 but substituting N-[3-(chloromethyl)-4-cyclopentylphenyl]-2-thiophenecarboximidamide hydrochloride for N-[3-(chloromethyl)-4-(1-ethylpropoxy)phenyl)]-2-thiophenecarboximidarride hydrochloride the title compound was obtained as a colourless solid in 76% yield; m.p. 116-118° C.; MS: ^{m}/z 358 [M+H]⁺.

### EXAMPLE 31

N-(4-Cyclopentyl-3-{[(2S)-2-(Hydroxymethyl)Pyrrolidinyl]Methyl}Phenyl)-2-Thiophenecarboximidamide

Using the method described in Example 12 but substituting (S)-(+)-2-pyrrolidinemethanol for 2-(methylamino)ethanol and substituting N-[3-(chloromethyl)-4-cyclopentylphenyl]-2-thiophenecarboximidamide hydrochloride for N-[3-(chloromethyl)-4-(1-ethylpropoxy)phenyl)]-2-thiophenecarboximidamide hydrochloride the title compound was obtained as a colourless solid in 80% yield; m.p. 132-133° C.; MS: ^{m}/z 384 [M+H]⁺.

### EXAMPLE 32

N-(4-Cyclopentyl-3-{[(2R)-2-(Hydroxymethyl)Pyrrolidinyl]Methyl}Phenyl)-2-Thiophenecarboximidamide

Using the method described in Example 12 but substituting (R)-(−)-2-pyrrolidinemethanol for 2-(methylamino)ethanol and substituting N-[3-(chloromethyl)-4-cyclopentylphenyl]-2-thiopenecarboximidamide hydrochloride for N-[3-(chlorormethyl)-4-(1-ethylpropoxy)phenyl)]-2-thiophenecarboximidamide hydrochloride the title compound was obtained as a colourless solid in 90% yield; m.p. 130-132° C.; MS: ^{m}/z 84 [M+H]⁺.

### EXAMPLE 33

N-(3-{[(2 -Hydroxyethyl)(Methyl)Amino]Methyl}-4-Methoxyphenyl)-3-Thiophenecarboximidamide

Using the method described in Example 1(c) but substituting 3-thiophenecarboximidothioic acid ethyl ester hydrochloride for 2-thiophenecarboximidothioic acid ethyl ester hydrochloride and substituting 2-[(5-amino-2-methoxybenzyl)(methly)aminolethanol hydrochloride for [(2R)-1-(5-amino-2-methoxybenzyl)pyrrolidinyl]methanol hydrochloride the title compound (as free base) was obtained as a colourless solid in 15% yield; MS: ^{m}/z 320 [M+H]⁺.

### EXAMPLE 34

N-(3-{[(2-Hydroxyethyl)Amino]Methyl}-2-Methylphenyl)-2-Thiophenecarboximidamide Dihydrochloride

To a stirred solution containing N-(3-chloromethyl-2-methylphenyl)thiophene-2-carboxamidine hydrochloride (1.00 g, 2.97 mmol) in DMF (5 mL) was added ethanolamine (893 μL, 14.8 mmol) and dilsopropylethylamine (569 μL, 3.27 mmol). The solution stirred for 5 h, then the DMF was removed under reduced pressure at 60° C. The residue was treated with water (10 mL) then basified to pH 11 with 1N sodium hydroxide solution. The aqueous layer was extracted with ethyl acetate (10 mL) then the organic was washed with water (3×10 mL) and brine (1×10 mL). The organic layer was then dried (sodium sulphate), filtered and concentrated under reduced pressure to give a crude oil. Purification by silica gel column chromatography using a dichloromethane:methanol gradient afforded the free base of the title compound as an oil/solid. The free base was dissolved in dichloromethane and acidified with hydrogen chloride in diethyl ether to afford the title compound (400 mg, 37%) as a white solid: MS: ^{m}/z 290 [M−H]⁺.

### EXAMPLE 35

N-(3-{[(2-Hydroxyethyl)(Methyl)Amino]Methyl}-2-Methylphenyl)-2-Thiophenecarboximidamide Dihydrochloride

Using the method described in Example 34 but without purification by silica gel column chromatography and substituting 2-(methylamino)ethanol for ethanolamine, the title compound was obtained as a white solid in 99% yield; MS: ^{m}/z 304 [M+H]⁺.

### EXAMPLE 36

N-(3-{[(2R)-2-(Hydroxymethyl)Pyrrolidinyl]Methyl}-2-Methylphenyl)-2-Thiophenecarboximidamide Dihydrochloride

Using the method described in Example 34 but without purification by silica gel column chromatography and substituting R-(−)-2-pyrrolidinemethanol for ethanolamine, the title compound was obtained as a white solid in 98% yield; MS: ^{m}/z 330 [M+H]⁺.

### EXAMPLE 37

N-(5-{[(2-Hydroxyethyl)(Methyl)Amino]Methyl}-2-Methylphenyl)-2-Thiophenecarboximidamide Dihydrochloride

a) N-(5-Chloromethyl-2-Methylphenyl)-2-Thiophenecarboximidamide Hydrochloride

A mixture of 3-amino-4-methylbenzyl alcohol (10.0 g, 72.9 mmol) and 2-thiophenecarboximidothioic acid ethyl ester hydrochloride (22.7 g, 109 mmol) in ethanol (150 mL) was heated to reflux for 6 h. Solvents were evaporated to yield an orange liquid. To a mixture of the orange liquid in dichloromethane (190 mL) was added thionyl chloride (10.6 mL, 145.8 mmol) slowly. The reaction was stirred at room temperature for 3 h. Solvents were then removed to afford a dark yellow residue. The mixture was triturated with ether (2×200 mL) and a semi-solid was collected by vacuum filtration. This material was washed with ether and dried in vacuo to afford a light yellow solid (25.4 g); MS: ^{m}/z 265, 267 [M+H]⁺.

(b) N-(5-{[(2-Hydroxyethyl)(Methyl)Amino]Methyl}-2-Methylphenyl)-2-Thiophenecarboximidamide Dihydrochloride

A mixture of 2-(5-chloromethyl-2-methylphenyl)-2-thiophenecarboximidamide hydrochloride (1.20 g) and 2-(methylamino)ethanol (643 μL, 8 mmol) in acetonitrile (6 mL) was stirred at room temperature for 5 h. Water was added and the product was extracted with dichloromethane. The organic extracts were dried over sodium sulphate and removal of solvents yielded a liquid. Silica gel chromatography (20:1 dichloromethane/2M ammonia in methanol then 10:1 dichloromethane/2M ammonia in methanol) yielded a colourless liquid. This was taken up in methanol and treated with an excess of 1N hydrogen chloride in diethyl ether. Removal of solvents followed by drying in vactio afforded the title compound (247 mg) as a white solid; MS: ^{m}/z 304 [M+H]⁺.

### EXAMPLE 38

N-(3-{1-[(2-Hydroxyethyl)Amino]Ethyl}-4-Methoxyphenyl)-2-Thiophenecarboximidamide

N-(3-Acetyl-4-methoxphenyl)-2-thiophenecarboximidaTnide (443 mg, 1.62 mmol) was dissolved in a solution prepared by adding acetic acid (1.89 ml, 1.91 g, 31.3 mmol) to a solution of ethanolamine (1.82 ml, 1.83 g, 30 mnmol) in methanol (15 ml). After about 4 h, sodium cyanoborohydride (104 mg, 1.65 mmol) was added, and stirring was continued for 3 days. The solution was evaporated. In a fume hood, hydrochloric acid was added to the residue. After about 1 h, the solution was basified, first with solid potassium carbonate followed by aqueous sodium hydroxide, then extracted with dichloromethane. The organic extract was dried (magnesium sulfate), filtered, and evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the dihydrochloride salt of the title compound as a solid (172 mg, 0.44 mmol, 27%). MS (ES⁺) ^{m}/z 320 (100%, MH⁻).

### EXAMPLE 39

N-(3-{1-[(2-Hydroxyethyl)(Methyl)Amino]Ethyl}-4-Methoxyphenyl)-2-Thionhenecarboximidamide

N-(3-Acetyl-4-methoxyphenyl)-2-thiophenecarboximidamide (404 mg, 1.47 mmol) was dissolved in a solution prepared by adding acetic acid (1.89 ml, 1.91 g, 31.3 mmol) to a solution of 2-(N-methylamino)ethanol (2.42 ml, 2.26 g, 30 mmol) in methanol (15 ml). After about 4 h, sodium cyanoborohydride (111 mg, 1.78 mmol) was added, and stirring was continued for 3 days. The solution was evaporated. In a fume hood, hydrochloric acid was added to the residue. After about 1 h, the solution was basified, first with solid potassium carbonate followed by aqueous sodium hydroxide, and then extracted with dichloromethane. The organic extract was dried (magnesium sulfate), filtered, and evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the dihydrochloride salt of the title compound as a solid (214 mg, 0.53 mmol, 36%). MS (ES⁻) ^{m}/z 334 (100%, MH⁻).

### EXAMPLE 40

N-{3-[1 -(2-Hydroxyethyl)-2-Pyrrolidinyl]-4-Methoxyphenyl]-2-Thiophenecarboximidamide

(a) 4-Bromo-2-[1-(2-Hydroxyethyl)-2-Pyrrolidinyl]Phenyl Methyl Ether

Ethanolamine (5.2 ml, 5.28 g, 86.5 mmol) and acetic acid (5.3 ml, 5.55 g, 92.6 mmol) were added to a solution of 1-(5-bromo-2-methoxyphenyl)-4-chloro-1-butanone (5.00 g, 17.1 mmol) in methanol (50 ml). After about 1 h, sodium cyanoborohydride (1.61 g, 25.6 mmol) was added, and stirring was continued overnight. The solution was evaporated. In a fume hood, hydrochloric acid was added to the residue. After about 1 h, the solution was basified with aqueous sodium hydroxide, and then extracted with dichloromethane. The organic extract was dried (magnesium sulfate), filtered, and evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the hydrochloride salt of the sub-title compound as a solid (1.20 g, 3.57 mmol, 20%). MS (ES⁻) ^{m}/z 300, 302 (MH⁻).

(b) N-{3-[1-(2-Hydroxyethyl)-2-Pyrrolidinyl]-4-Methoxyphenyl]-2-Thiophenecarboximidamide

The hydrochloride salt of 4-bromo-2-[1-(2-hydroxyethyl)-2-pyrrolidinyl]phenyl methyl ether (1.20 g, 3.57 mmol) was converted into the free base by partitioning between aqueous sodium hydroxide and dichloromethane. The organic layer was dried with magnesium sulfate, then evaporated and dried under vacuum. In a dry tube, a solution of the 4-bromo-2-[1-(2-hydroxyethyl)-2-pyrrolidinyl]phenyl methyl ether free base in dry tetrahydrofuran (25 ml) was added to a mixture of tris(dibenzylidineacetone)dipalladium (0) (39 mg, 0.043 mmol), racemic 2,2′-bis(diphenylphosphino)-1,1′-binaphthyl (33 mg, 0.133 mmol), and sodium t-butoxide (1.12 g, 11.6 mmol). Benzophenone imine (1.00 ml, 1.08 g, 6.0 mmol) was added, and the tube was then sealed under nitrogen. The solution was then stirred at 100° C. (bath temperature) overnight. The solution was then allowed to cool, and was evaporated. The residue was partitioned between aqueous sodium carbonate and dichloromethane, and the organic layer was dried (magnesium sulfate), filtered, and evaporated. The residue was dissolved in tetrahydrofiuran (50 ml) and 6N hydrochloric acid (5 ml) was added. After 5 h, the solution was evaporated, the residue was partitioned between aqueous sodium carbonate and dichloromethane, and the organic extract was dried (magnesium sulfate), filtered, and evaporated. To the residue, 2-thiophenecarboximidothioic acid ethyl ester hydrochloride (2.49 g, 11.98 mmol), ethanol (30 ml), and triethylamine (5 ml) were added, and the solution was heated under nitrogen at 80° C. (bath temperature) ovemright. The solution was evaporated, the residue was partitioned between sodium carbonate and chloroform, and the organic extract was dried (magnesium sulfate), filtered, and evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the dihydrochloride salt of the title compound as a solid (45.7 mg, 0.109 mmol, 3%). MS (ES⁺) ^{m}/z 346 (100%, MH⁻).

### EXAMPLE 41

N-(3-(2-[Benzyl-(2-Hydroxyethyl)Amino]Ethyl)Phenyl)-2-Thiophenecarboximidamide

MS (ES⁺) ^{m}/z 380 (MH⁻).

### EXAMPLE 42

N-(3-([Benzyl-(2-Hydroxyethyl)Amino]Methyl)Phenyl)-2-Thiophenecarboximidamide

MS (ES⁺) ^{m}/z 366 (MH⁻).

### EXAMPLE 43

N-(3-(2-[(1-Hydroxmethyl)Butylamino]Ethyl)Phenyl)-2-Thiophenecarboximidamide

MS (ES⁺) ^{m}/z 332 (100%, MH⁺).

1. A compound of formula (I) 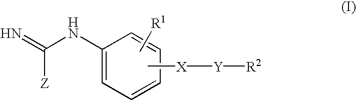
wherein
Z represents a furan or thiophene ring, optionally substituted by one or more substituents selected from halogen. trifluoromethyl, C1 to 6 alkyl, C1 to 6 alkoxy, hydroxy, amino, S(O)_{q}R⁴, CO₂R⁵ and CONR⁶R⁷;
X represents C1 to 6 alkyl;
Y represents O, S(O)ₙ or NR³;
n and q independently represent an integer 0, 1 or 2;
R¹ represents hydrogen, halogen, C1 to 6 alkyl, hydroxy, C1 to 6 alkoxy,
C1to 6 alkoxy-O—R⁸, C1 to 6 alkoxy-NR⁹R¹⁰ or —O-phenyl; said phenyl being optionally substituted by one or more substituents selected from halogen, trifluoromethyl, C1 to 6 alkyl, C1 to 6 alkoxy, hydroxy and amino;
R² represents C1 to 6 alkyl-O—R¹¹ or C1to 6 alkyl-NR¹²R¹³;
R³ represents hydrogen, C1 to 6 alkyl, C2 to 7 alkanoyl, C1 to 6 alkyl-O—R¹⁴, C1 to 6 alkyl-NR¹⁵R¹⁶ or —CH₂-phenyl; said phenyl being optionally substituted by one or more substituents selected from halogen, trifluoromethyl, C1 to 6 alkyl, C1 to 6 alkoxy, hydroxy and amino;
or the group NR²R³ represents azetidinyl, pyrrolidinyl, piperidinyl, morpholinyl; or piperazinyl optionally-substituted by C1 to 6 alkyl; each of said azacyclic rings being substituted by O—R¹⁷, NR¹⁸R¹⁹, C1 to 6 alkyl-O—R¹⁷ or C1 to 6 alkyl-NR¹⁸R¹⁹;
or, when Y represents NR³, the groups X and R³ are joined together such that the group X—N—R³ represents a saturated 4 to 7 membered azacyclic ring;
R⁴, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ independently represent hydrogen or C1 to 6 alkyl;
or the groups NR⁹R¹⁰, NR¹²R¹³, NR¹⁵R¹⁶ and NR¹⁸R¹⁹ independently represent azetidinyt, pyrrolidinyl, piperidinyl, morpholinyl; or piperazinyl optionally 4-substituted by C1 to 6 alkyl;
and optical isomers, racemates and tautomers thereof and pharmaceutically acceptable salts thereof.

2. A compound of formula (I), according to claim 1, wherein the substituent R¹ in formula (I) is in the para position relative to the amidine group.

3. A compound of formula (I), according to either of claims 1 or 2, wherein the substituent —X—Y—R² in formula (I) is in the meta position relative to the amidine group.

4. A compound of formula (I), according to any one of claims 1 to 3, wherein Y represents NR³.

5. A compound of formula (I), according to any one of claims 1 to 4, wherein the group R¹ represents methioxy.

6. A compound of formula (I), according to any one of claims 1 to 5, wherein X represents CH₂.

7. A compound of formula (I), according to claim 1, which is:
N-(3-{[(2R)-2-(hydroxymethyl)pyrrolidinyl]methyl}-4-methoxyphenyl)-2-thiophenecarboximidamnide;
N-(3-{[(2S)-2-(hydroxymethyl)pyrrolidinyl]methyl}-4-methoxyphenyl)-2-thiophenecarboximidamide;
N-(3-{[(2-hydroxyethyl)(methyl)amino]methyl}-4-methoxyphenyl)-9-thiophenecarboximidamide;
N-[3-({[1-(hydroxymethyl)butyl]amino}methyl)-4-methoxyphenyl]-2-thiophenecarboximidamide;
N-(3-{[(2-hydroxyethyl)(methyl)amino]methyl}phenyl)-2-thiophenecarboximidamide;
N-[3-{[1-(hydroxymethyl)butyl]amino}methyl)phenyl]-2-thiophenecarboximidamide;
N-(3-{[hexyl(2-hydroxyethyl)amino}methyl]phenyl)-2-thiophenecarboximidamide;
N-(3-{[(2-hydroxyethyl)amino]methyl}-4-methoxyphenyl)-2-thiophenecarboximidamide;
N-(4-methoxy-3-{[(2-methoxyethyl)amino]methyl}phenyl-2-thiophenecarboximidamide:
N-(3-{[bis(2-hydroxyethyl)amino]methyl}4-methoxyphenyl)-2-thiophenecarboximidamide;
N-(3-{[(cyclopropyl)(2-hydroxyethyl)amino]methyl}-methoxy)-2-thiophenecarboximidamide;
N-(4-(1-ethylpropoxy)-3-{[(2-hydroxyethyl)(methyl)amino]methyl}phenyl)-2-thiophenecarboximidamide;
N-(4-(1-ethylpropoxy)-3-{[(2-hydroxyethyl)amino]methyl}phenyl)-2-thiophenecarboximidamide;
N-[3-{[(2-aminoethyl)amino]methyl}-4-(1-ethylpropoxy)phenyl]-2-thiophenecarboximidamide;
N-[4-(1-ethylpropoxy)-3-({[2-(1-piperazinyl)ethyl]amino}methyl)phenyl]-2-thiophenecarboximidamide;
N-(4-(1-ethylpropoxy)-3-{[(4-hydroxybutyl)amino]methyl}phenyl)-2-thiophenecarboximidamide;
N-(3-{[(2-hydroxyethyl)(methyl)amino]methyl}-4-isopropoxyphenyl)-2-thiophenecarboximidamide;
N-(3-{[(4-hydroxybutyl)amino]methyl}-4-isopropoxyphenyl)-2-thiophenecarboximidamide;
N-[4-isopropoxy-3-({[2-(1-piperazinyl)ethyl]amino}methyl)phenyl]-2-thiophenecarboximidamide;
N-(4-(cyclopentyloxy)-3-{[(2-hydroxyethyl)(methyl)amino]methyl}phenyl)-2-thiophenecarboximidamide;
N-(4-(cyclopentyloxy)-3-{[(2R)-2-(hydroxymethyl)pyrrolidinyl]methyl}phenyl)-2-thiophenecarboximidamide;
N-(3-{[(2-hydroxyethyl)(methyl)amino]methyl}-4-phenoxyphenyl)-2-thiophenecarboximidamide;
N-(3-{[(2R)-2-(hydroxymethyl)pyrrolidinyl]methyl}phenyl)-2-thiophenecarboximidamide;
N-(3-{[(2S)-2-(hydroxymethyl)pyrrolidinyl]methyl}phenyl)-2-thiophenecarboximidamide;
N-(3-{[(2-hydroxyethyl)amino]methyl}phenyl)-2-thiophenecarboximidamide;
N-(3-{[(2-methoxyethyl)amino]methyl}phenyl)-2-thiophenecarboximidamide;
N-(3-{[hexyl(2-hydroxyethyl)amino]methyl}-4-methoxyphenyl)-2-thiophenecarboximidamide;
N-(3-[3-hydroxypiperidinylmethyl]phenyl)-2-thiophenecarboximidamide;
N-(3-[4-hydroxypiperidinylmethyl]phenyl)-2-thiophenecarboximidamide;
N-(4-cyclopenty[-3-{[(2-hydroxyethyl)(methyl)amino]methyl}phenyl)-2-thiophenecarboximidamide;
N-(4-cyclopentyl-3-{[(2S)-2-(hydroxymethyl)pyrrolidinyl]methyl}phenyl)-2-thiophenecarboximidamide;
N-(4-cyclopentyl-3-{[(2R)-2-(hydroxymethyv)pyrrolidinyl]methyl}phenyl)-2-thiophenecarboximidamide;
N-(3-{[(2-hydroxyethyl)(methyl)amino]methyl}-4-methoxyphenyl)-3-thiophenecarboximidamide;
N-(3-{[(2-hydroxyethyl)amino]methyl}-2-methylphenyl)-2-thiophenecarboximidamide;
N-(3-{[(2-hydroxyethyl)(methyl)amino]methyl}-2-methylphenyl)-2-thiophenecarboximidamide;
N-(3-{[(2R)-2-(hydroxymethyl)pyrrolidinyl]methyl}-2-methylphenyl)-2-thiophenecarboximidamide;
N-(5-{[(2-hydroxyethyl)(methyl)amino]methyl}-2-methylphenyl)-2-thiophenecarboximidamide;
N-(3-{1-[(2-hydroxyethyl)amino]ethyl}-4-methoxyphenyl)-2-thiophenecarboximidamide;
N-(3-{1-[(2-hydroxyethyl)(methyl)amino]ethyl}-4-methoxyphenyl)-2-thiophenecarboximidamide;
N-{3-[1 -(2-hydroxyethyl)-2-pyrrolidinyl]-4-methoxyphenyl]-2-thiophenecarboximidamide;
N-(3-(2-[benzyl-(2-hydroxyethyl)amino]ethyl)phenyl)-2-thiophenecarboximidamide;
N-(3-([benzyl-(2-hydroxyethyl)amino]methyl)phenyl)-2-thiophenecarboximidamide;
N-(3-(2-[(1-hydroxymethyl)butylamino]ethyl)phenyl)-2-thiophenecarboximidamide;
or an optical isomer, racemate or tautomer of any one thereof or a pharmaceutically acceptable salt of any one thereof.

8. A compound of formula (I), as defined in any one of claims 1 to 7, for use as a medicament.

9. A pharmaceutical formulation compnrsing a compound of formula (I), as defined in any one of claims 1 to 7, or an optical isomer, racemate or tautomer thereof or a pharmaceutically acceptable salt thereof, optionally in admixture with a pharmaceutically acceptable diluent or carrier.

10. A method of treating, or reducing the risk of, a human disease or condition in which inhibition of nitric oxide synthase activity is beneficial which comprises administering to a person suffering from or susceptible to such a disease or condition, a therapeutically effective amount of a compound of formula (I), as defined in any one of claims 1 to 7, or an optical isomer, racemate or tautomer thereof or a pharmaceutically acceptable salt thereof.

11. A method of treatment according to claim 10 in which it is predominantly the neuronal isoform of nitric oxide synthase that is inhibited.

12. A method of treating, or reducing the risk of hypoxia or stroke or ischaemia or neurodegenerative conditions or schizophrenia or anxiety or pain or migraine, which comprises administering to a person suffering from or susceptible to such a disease or condition a therapeutically effective amount of a compound of formula (I), as defined in any one of claims 1 to 7, or an optical isomer, racemate or tautomer thereof or a pharmaceutically acceptable salt thereof.

13. A method of treatment according to claim 12, wherein the condition to be treated is selected from the group consisting of hypoxia, ischaemia, stroke, Parkinson's disease, anxiety, schizophrenia, migraine and pain.

14. A method of treatment according to claim 13, wherein the condition to be treated is stroke.

15. A method of treatment according to claim 13, wherein the condition to be treated is pain.

16. A method of treatment according to claim 13, wherein the condition to be treated is migraine.

17. A method of treatment according to claim 13, wherein the condition to be treated is schizophrenia.

18. A method of treatment according to claim 13, wherein the condition to be treated is Parkinson's disease

19. The use of a compound of formula (I) as defined in any one of claims 1 to 7, or an optical isomer, racemate or tautomer thereof or a pharaceutically acceptable salt thereof, in the manufacture of a medicament for the treatment or prophylaxis of human diseases or conditions in which inhibition of nitric oxide synthase activity is beneficial.

20. The use as claimed in claim 19 wherein it is predomninantly the neuronal isoform of nitric oxide synthase that is inhibited.

21. The use of a compound of formula (I) as defined in any one of claims 1 to 7, or an optical isomer, racemate or tautomer thereof or a pharmaceutically acceptable salt thereof, in the manufacture of a medicament for the treatment or prophylaxis of hypoxia or stroke or ischaemia or neurodegenerative conditions or schizophrenia or anxiety or pain or migraine.

22. The use as claimed in claim 21, wherein the condition is selected from the group consisting of hypoxia, ischaemia, stroke, Parkinson's disease, anxiety, schizophrenia, migraine and pain.

23. The use as claimed in claim 22, wherein the condition is stroke.

24. The use as claimed in claim 22, wherein the condition is pain.

25. The use as claimed in claim 22, wherein the condition is migraine.

26. The use as claimed in claim 22, wherein the condition is schizophrenia.

27. The use as claimed in claim 22, wherein the condition is Parkinson's disease.

28. A process for the preparation of a compound of formula (I), as defined in any one of claims 1 to 7, and optical isomers, racemates and tautomers thereof and pharmaceutically acceptable salts thereof, which comprises:
(a) reacting a corresponding compound of formula (II) or a salt thereof 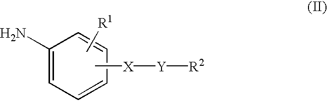
wherein R¹, R², X and Y are as defined in claim 1,
with a compound of formula (III) or a salt thereof 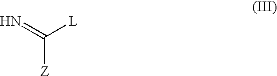
wherein Z is as defined in claim 1 and L represents a leaving group; or
(b) reacting a corresponding compound of formula (IV) or a salt thereof 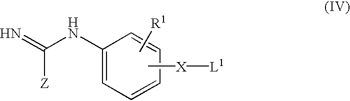
wherein R¹, X and Z are as defined in claim 1 and L¹ is a leaving group, with a compound of formula H—Y—R² or a salt thereof, wherein Y and R² are as defined in claim 1; or
(c) preparing a compound of formula (I) wherein X represents —CH₂— by reduction of a corresponding compound wherein X represents —CO— (formula V) 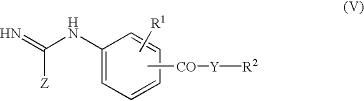
and where desired or necessary converting the resultant compound of formula (I), or another salt thereof, into a pharmaceutically acceptable salt thereof, or vice versa, and where desired converting the resultant compound of formula (I) into an optical isomer thereof.

29. A intermediate compound of formula (V) 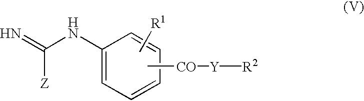
wherein R¹, R², Y and Z are as defined in claim 1.
